(19) United States
(12) Patent Application Publication
(10) Pub. No.: US 2015/0079686 A1
(43) Pub. Date: Mar. 19, 2015

(54) METHOD OF RESOLVING HEAVY METAL CONTAMINATION SOURCE BASED ON SEQUENTIAL EXTRACTION SCHEME AND ISOTOPE ANALYSIS SCHEME

(71) Applicant: KOREA INSTITUTE OF GEOSCIENCE AND MINERAL RESOURCES, Daejeon (KR)

(72) Inventor: Pyeong-Koo LEE, Daejeon (KR)

(73) Assignee: KOREA INSTITUTE OF GEOSCIENCE AND MINERAL RESOURCES, Daejeon (KR)

(21) Appl. No.: 14/201,647

(22) Filed: Mar. 7, 2014

(30) Foreign Application Priority Data
Sep. 13, 2013 (KR) 10-2013-0110098

(51) Int. Cl. G01N 33/20

(52) U.S. Cl. CPC G01N 33/20
USPC 436/73

(57) ABSTRACT

Disclosed is a method of resolving a heavy metal contamination source based on a sequential extraction scheme and an isotope analysis scheme. Pb isotopes are eluted at 5 types of “cation exchange fraction”, “carbonate fraction”, “iron-oxide and manganese hydroxide-fraction”, “organic matters and sulfide fraction”, and “residual fraction” existing at other types and separated from each other in each step. The Pb isotopes are analyzed and the origins of the Pb isotopes are specified in each step. The contamination source of the Pb isotope including ²⁰⁴Pb, ²⁰⁶Pb, and ²⁰⁷Pb is specified from a correlation between a ratio of ²⁰⁶Pb/²⁰⁴Pb obtained from a content of ²⁰⁶Pb and a content of ²⁰⁴Pb and a ratio of ²⁰⁶Pb/²⁰⁷Pb obtained from a content of ²⁰⁶Pb and a content of ²⁰⁷Pb in the Pb isotope.

### CROSS-REFERENCE TO RELATED APPLICATION

This application claims the benefit under 35 U.S.C. §119 of Korean Patent Application No. 10-2013-0110098 filed on Sep. 13, 2013 in the Korean Intellectual Property Office, the entirety of which disclosure is incorporated herein by reference.

### BACKGROUND OF THE INVENTION

1) Field of the invention

The present invention relates to a method of resolving a heavy metal contamination source. In more detail, the present invention relates to a method of resolving a heavy metal contamination source based on a sequential extraction scheme and an isotope analysis scheme, capable of exactly analyzing and finding the contamination source of heavy metal harmful to a human body.

2) Background of Related Art

An industrial revolution is progressed since 18C, and a huge amount of heavy metal is discharged into a natural environment as a result of the industrial revolution.

In addition, to support the industrial revolution, many metallic resources including heavy metal are mined for the industrial use and wasted.

In this case, the wasted results of the industrial activity contaminate the natural environment through several paths. When mines are abandoned, harmful heavy metal is continuously discharged and environment pollution is caused.

Therefore, it is necessary to prevent additional environmental pollution by detecting the source of the heavy metal when natural-state heavy metal is spread or moved, or exactly detecting the source of the heavy metal discharged from the abandoned mine.

Meanwhile, Korea is affected by Asian dust come from China throughout winter and spring. The Asian dust come from China is mixed with other contamination sources resulting from the industrial activity in China and moved into Korea and Japan.

The contamination materials such as heavy metal resulting from the industrial activity and the Asian dust cross the boundary between nations and move a long distance. Accordingly, since the contamination materials and the Asian dust may serve as broad contamination sources between nations to cause conflicts between related nations.

Accordingly, each related nation makes an effort to detect the exact source of the heavy metal contamination source.

Recently, among attempts to detect the heavy metal contamination sources, study and researches have been extensively performed regarding a technology to investigate the heavy metal contamination sources by using lead (Pb) isotopes.

In this case, the Pb exists as isotopes of ²⁰⁴Pb, ²⁰⁶Pb, ²⁰⁷Pb, and ²⁰⁸Pb. Among them, only ²⁰⁴Pb exists as Pb in a stable state since the creation of earth, and remaining isotopes are generally known as radiogenic isotopes created through the radioactive decay of ²³⁸U, ²³⁵U, and ²³²Th.

In general, since Pb rarely includes U or Th, it may be assumed that the content of U or Th is actually maintained without change when a Pb contamination source is created.

Accordingly, if the ratio between Pb isotopes contained in a Pb contamination source is traced, the origins of Pb contamination sources may be investigated.

In detail, when a soil (including sedimentary layers) is formed, a soil mixed through a weathering process of several grounds instead of a soil formed through the weathering process of the ground having the same origin may be estimated to represent a value varied depending on the mixture ratio.

Similarly, even if a Pb isotope resulting from the activity of a human being, that is, the industrial activity is added to the soil formed through the weathering process of a pure single ground, the content of the Pb isotope, which is finally analyzed, represents a mixed value of the content of the Pb isotope derived from the pure single ground and the content the Pb isotope derived from an artificial contamination source according to the content ratios of the Pb isotopes.

In other words, the Pb isotopes having various origins are expected to represent the difference in the content between mutually different Pb isotopes according to the content ratios of the Pb isotopes when comparing with the content of the Pb isotope formed from the pure single ground.

Meanwhile, up to now, in order to analyze Pb isotopes in an environmental pollution analysis field, a total content analysis scheme, which is known as a full decomposition analysis scheme, has been used

The full decomposition analysis scheme is to analyze whole samples including a soil contaminated with heavy metal, deposits, dust, and non-Asian dust.

In other words, the full decomposition analysis scheme is to chemically analyze the whole samples to be analyzed in the pre-treatment of the samples.

However, the total content analysis scheme does not reflect the characteristics, such as a mineral characteristic, the content of the organic matters, and physical and chemical characteristics, of a specific Pb isotope existing in the samples in relation to the content of Pb existing in a sample to be analyzed.

The whole Pb content in the sample has been analyzed, and the characteristic of the Pb isotope has been detected by using the analysis result without the determination if the Pb isotope is derived from a elastic mineral such as a primary mineral, a secondary mineral, or a tertiary mineral, or without the distinguishment between the secondary mineral and the tertiary mineral serving as an artificial contamination source of the Pb isotope resulting from the activity of a human being.

When the content of the Pb isotope is analyzed with respect to the sample treated through the total content analysis scheme, the primary mineral, the secondary mineral, and the tertiary mineral and the Pb fractioned from the above minerals may be mixed with each other. Therefore, the Pb isotope is analyzed in each step in the mixed state with the minerals without the effective separation of the contamination minerals, so that the exact analysis of the origin of the Pb isotope in the mixed state is difficult.

Although reference document 1 (Suh Ji-Won, Yoon Hye-On, and Jeong Chan-Ho, “The Distribution Characteristics and Contamination of Heavy Metals in Soil from Dalcheon Mine” in Journal of the Mineralogical Society of Korea, Vol. 21, No. 1, p. 57-65, March, 2008) among cited references employs a sequential extraction scheme to detect the type of heavy metal existing in a soil and the contamination degree of the heavy metal, only the content of the heavy metal contained in the soil is detected.

Further, although reference document 2 (Choi Man-Sik, Cheong Chang-Sik, Han Jeong-Hee, and Park Kye-Hun, “Distribution and Sources of Pb in Southern East/Japan Sea Sediments using Pb isotopes”, Economic and Environmental Geology, Vol. 39, No. 1, p. 63-74, 2006) employs the Pb isotope in order to investigate the origins of the Pb existing in the deposits, reference document 2 discloses the total content analysis scheme to put a sample in HCl and HNO₃ solution and elute the sample.

### SUMMARY OF THE INVENTION

One object of the present invention is to provide a method of exactly specifying a contamination source of heavy metal harmful to a human body.

Another object of the present invention is to exactly specify an artificial contamination source of a Pb isotope resulting from the industrial activity of a human being.

The objects of the present invention are not limited to the above-mentioned objects, and other objects will be clearly understood by those skilled in the art.

In order to accomplish the above object, there is provided a method of resolving a heavy metal contamination source based on a sequential extraction scheme and an isotope analysis scheme. The method includes (A) preparing a first sample including a Pb isotope, (B) preparing a first solution including 1M MgCl₂ (pH=7), introducing the first sample into the first solution, and stirring the first solution at a normal temperature for one hour to retrieve a second solution, (C) preparing a third solution including 1M CH₃COONa, adjusting a pH of the third solution to 5 by using HOAc, introducing a second sample, and stirring a result at the normal temperature for five hours to obtain a fourth solution and retrieve a third sample which is not dissolved, (D) preparing a fifth solution including 0.04M NH₂OH.HCl+25% HOAc, adjusting a pH of the fifth solution to 2, introducing the third sample, and heating the result at the temperature of 96° C. for six hours to obtain a sixth solution and retrieve a fourth sample which is not dissolved, (E) preparing a seventh solution including 30% H₂O₂+0.02M HNO₃, introducing the fourth sample, heating a result at the temperature of 85° C. for five hours, cooling the result, additionally introducing an eighth solution including 3.2M NH₄OAc+20% HNO₃, and stirring a result at the normal temperature for 30 minutes to obtain a ninth solution, (F) introducing the fifth sample into a tenth solution including HF+HClO₄, evaporating a result at a temperature of 110° C. to completely dry the result, introducing the result into 12M HCl solution, heating the result for 30 minutes, and completely dissolving the fifth sample to obtain an eleventh solution, and (G) analyzing a content of the Pb isotope contained in a Pb isotope effluent of the second solution, the fourth solution, the sixth solution, the ninth solution, and the eleventh solution obtained in steps (B) to (F).

In this case, preferably, the Pb isotope includes ²⁰⁴Pb, ²⁰⁶Pb, ²⁰⁷Pb, and ²⁰⁸Pb. Among them, the Pb isotope used in the analyzing of the content of the Pb isotope in step (G) includes and ²⁰⁷Pb, ²⁰⁶Pb, and ²⁰⁷Pb.

In addition, preferably, a contamination source of the Pb isotope including ²⁰⁴Pb, ²⁰⁶Pb, and ²⁰⁷Pb is specified from a correlation between a ratio of ²⁰⁶Pb/²⁰⁴Pb obtained from a content of ²⁰⁶Pb and a content of ²⁰⁴Pb and a ratio, ²⁰⁶Pb/²⁰⁷Pb, between a content of ²⁰⁶Pb and a content of ²⁰⁷Pb in the Pb isotope.

Preferably, the sample is pulverized in a particle size of 80 meshes to 100 meshes.

In addition, there is provided a method of resolving a heavy metal contamination source based on a sequential extraction scheme and an isotope analysis scheme. The method includes (A) preparing a first sample including a Pb isotope, (B) preparing a first solution including 1M MgCl₂ (pH=7), introducing the first sample into the first solution, and stirring the first solution at a normal temperature for one hour to retrieve a second solution, (C) preparing a third solution including 1M CH₃COONa, adjusting a pH of the third solution to 5 by using HOAc, introducing a second sample, and stirring a result at the normal temperature for five hours to obtain a fourth solution and retrieve a third sample which is not dissolved, (D) preparing a fifth solution including 0.04M NH₂OH.HCl+25% HOAc, adjusting a pH of the fifth solution to 2, introducing the third sample, and heating a result at the temperature of 96° C. for six hours to obtain a sixth solution and retrieve a fourth sample which is not dissolved, (E) preparing a seventh solution including 30% H₂O₂+0.02M HNO₃, introducing the fourth sample, heating a result at the temperature of 85° C. for five hours, cooling the result, additionally introducing an eighth solution including 3.2M NH₄OAc+20% HNO₃, and stirring a result at the normal temperature for 30 minutes to obtain a ninth solution, and (F) analyzing a content of the Pb isotope contained in a Pb isotope effluent of the sixth solution obtained in step (D) or the ninth solution obtained in step (E). A Pb isotope contamination source resulting from an industrial activity is specified in step (F).

In this case, preferably, the Pb isotope includes ²⁰⁴Pb, ²⁰⁶Pb, ²⁰⁷Pb, and ²⁰⁸Pb. Among them, the Pb isotope used in the analyzing of the content of the Pb isotope in step (G) includes ²⁰⁴Pb, ²⁰⁶Pb, and ²⁰⁷Pb.

In addition, preferably, a contamination source of the Pb isotope including ²⁰⁴Pb, ²⁰⁶Pb, and ²⁰⁷Pb is specified from a correlation between a ratio of ²⁰⁶Pb/²⁰⁴Pb obtained from a content of ²⁰⁶Pb and a content of ²⁰⁴Pb and a ratio of ²⁰⁶Pb/²⁰⁷Pb obtained from a content of ²⁰⁶Pb and a content of ²⁰⁷Pb in the Pb isotope.

Preferably, the sample is pulverized in a particle size of 80 meshes to 100 meshes.

The details of other embodiments are included in the following description and accompanying drawings.

The advantages, the features, and schemes of achieving the advantages and features of the present invention will be apparently comprehended by those skilled in the art based on the embodiments, which are detailed later in detail, together with accompanying drawings. The present invention is not limited to the following embodiments but includes various applications and modifications. The embodiments will make the disclosure of the present invention complete, and allow those skilled in the art to completely comprehend the scope of the present invention. The present invention is only defined within the scope of accompanying claims.

Those skilled in the art should comprehend that the same reference numerals will be assigned to the same elements in the following description, and the sizes, the positions, and the coupling relationship of components will be partially exaggerated for clarity.

As described above, according to the present mention, the contamination source of Keay metal, in detail, Pb can be exactly analyzed by simultaneously performing the sequential extraction scheme and the isotope analysis scheme. Therefore, the contamination source of Pb can be exactly specified.

In addition, according to the present invention, an artificial contamination source of a Pb isotope resulting from the industrial activity of human beings can be specified.

### BRIEF DESCRIPTION OF DRAWINGS

FIG. 1 is a flowchart schematically showing the sequence of a method of resolving a heavy metal contamination source based on a sequential extraction scheme and an isotope analysis scheme according to an exemplary embodiment of the present invention.

FIGS. 2A to 2E are graphs showing analysis results according to the method of resolving the heavy metal contamination source based on the sequential extraction scheme and the isotope analysis scheme according to the exemplary embodiment of the present invention.

FIGS. 3A to 3E are graphs showing analysis results according to the first step in the method of resolving the heavy metal contamination source based on the sequential extraction scheme and the isotope analysis scheme according to the exemplary embodiment of the present invention.

FIGS. 4A to 4E are graphs showing analysis results according to the second step in the method of resolving the heavy metal contamination source based on the sequential extraction scheme and the isotope analysis scheme according to the exemplary embodiment of the present invention.

FIGS. 5A to 5E are graphs showing analysis results according to the third step in the method of resolving the heavy metal contamination source based on the sequential extraction scheme and the isotope analysis scheme according to the exemplary embodiment of the present invention.

FIGS. 6A to 6E are graphs showing analysis results according to the fourth step in the method of resolving the heavy metal contamination source based on the sequential extraction scheme and the isotope analysis scheme according to the exemplary embodiment of the present invention.

FIGS. 7A to 7E are graphs showing analysis results according to the fifth step in the method of resolving the heavy metal contamination source based on the sequential extraction scheme and the isotope analysis scheme according to the exemplary embodiment of the present invention.

FIGS. 8A to 8E are graphs showing an average value of the content ratios of Pb isotopes contained in Asian dust, non-Asian dust, and China desert soil in each step of the method of resolving the heavy metal contamination source based on the sequential extraction scheme and the isotope analysis scheme according to the exemplary embodiment of the present invention.

### DETAILED DESCRIPTION OF THE INVENTION

Hereinafter, a method of resolving a heavy metal contamination source based on a sequential extraction scheme and an isotope analysis scheme according to an exemplary embodiment of the present invention will be described in detail with reference to accompanying drawings.

First, the background to the present invention will be described below.

Although a soil or a sedimentary layer formed through a weathering process of a single ground has Pb existing in various physical or chemical shapes inside the soil or the sedimentary layer in the state that different contamination sources are mixed, the origins of the Pbs are substantially identical to each other.

However, the origins of the contents of Pb isotopes contained in a sample, in which all Pb isotopes come from various contamination sources having mutually different origins are mixed, may not be detected as described above.

Therefore, the content of the Pb isotope is analyzed in each step through various chemical processes, and the content of the Pb isotope of the soil formed from a pure single ground and the difference in the content between the Pb isotopes come from the contamination sources having different origins or different beginnings may be determined based on the analyzed content of the Pb isotope.

Although chemical treatment is repeated without the types of Pb in the case of a soil weathered from a ground subject to the same petrologic differentiation process, the ratio of Pb isotopes according to the types of the Pb isotope existing in each sample is the same.

Therefore, in the case of soils weathered from several different grounds subject to different petrologic differentiation processes or soils having Pb contaminants artificially generated from the result of an industrial activity, the difference in the ratio of Pb isotopes according to the types of the Pb isotopes may be made.

While keeping in the mind the background described above, the inventors of the present invention find a scheme of analyzing the content of a Pb isotope formed from a pure single ground is analyzed, and analyzing the contents of Pb isotopes, which can be obtained from contamination sources having various origins to analyze the origin of the contamination source.
1. Sample Preparation

Samples used in the present invention include Asian dust and non-Asian dust (see table 1).

The samples include Asian dust and Non Asian dust, and are collected by using a sample collecting tray made of stainless steel in a rooftop of the second research center in Korea Institute of Geoscience And Mineral Resources.

Six Asian dust samples are collected and two non Asian dust samples are collected.

In addition, seven desert soil samples (representative samples collected from four deserts of Taklimakan, Red clay, Ordos, and Alashan deserts) are prepared in order to compare with the Asian dust samples and the non Asian dust samples in the Pb isotope analysis.

In this case, the prepared desert soil sample is a China desert soil sample in China that is not contaminated from an external contamination source. If the content of the Pb isotope of the sample is calculated and the calculated content of the Pb isotope is set to a standard content of the Pb isotope, the contamination source of heavy metal existing in a sample can be specified by measuring the content of a Pb isotope in a sample, such as Asian dust or non Asian dust, which is collected in Korea, having high contamination possibility in a different contamination source and comparing the standard content of the Pb isotope with the sample collected in Korea.

2. Extraction of Pb Isotope Based on Sequential Extraction Scheme

A Pb isotope was extracted from the above prepared sample in each step through a sequential extraction scheme serving as a chemical pre-treatment scheme.

The sequential extraction scheme of the Pb isotope in each step will be described in detail below.

3. Analysis of Content of Pb isotope extracted through Sequential Extraction Scheme

The content of a Pb isotope existing in a Pb isotope effluent extracted through a sequential extraction scheme was measured.

The content of the Pb isotope was analyzed by Korea Basic Science Institute, and the technical description of the analysis of the content of the Pb isotope is omitted because the technical description is beyond the scope of the present invention.

4. Sequential Extraction Scheme

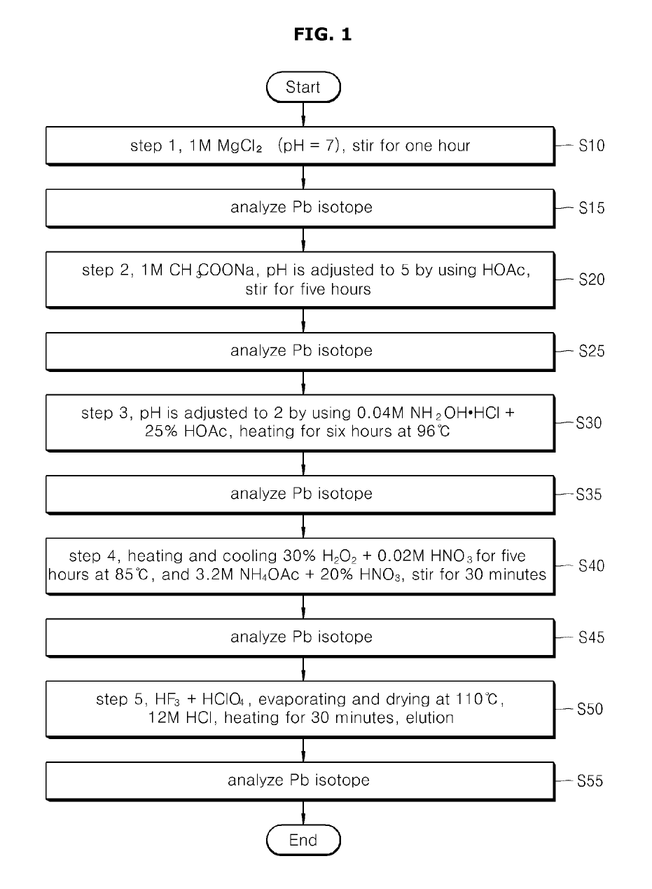

The sequential extraction scheme according to an exemplary embodiment of the present invention will be described with reference to FIG. 1.

FIG. 1 is a flowchart schematically showing the sequence of a method of resolving a heavy metal contamination source based on a sequential extraction scheme and an isotope analysis scheme according to an exemplary embodiment of the present invention.

As shown in FIG. 1, the sequential extraction scheme includes a step of preparing a first sample including a Pb isotope, preparing a first solution of 1M MgCl₂ (pH=7), introducing the first sample into the first solution, and stirring the first solution for one hour to obtain a second solution (step S10), a step of preparing a third solution of 1M CH₃COONa, adjusting the pH of the third solution to 5 by using HOAc, introducing a second sample retrieved in step S10, which is not dissolved, and stirring the result at a normal temperature for five hours to obtain a fourth solution (step S20), a step of preparing a fifth solution of 0.04M NH₂OH.HCl+25% HOAc, adjusting the pH of the fifth solution to 2, introducing the third sample retrieved in step S20, which is not dissolved, and heating the result for six hours at the temperature of 96° C. to obtain the sixth solution (step S30), a step of preparing a seventh solution of 30% H₂O₂+0.02M HNO₃, adjusting the pH of the fifth solution to 2, introducing the fourth sample retrieved in step S30, which is not dissolved, heating the result for five hours at the temperature of 85° C.; cooling the result, additionally introducing an eighth solution of 3.2M NH₄OAc+20% HNO₃, and stirring the result at the normal temperature for 30 minutes to obtain a ninth solution (step S40), and a step of introducing the fifth sample retrieved in step S40, which is not dissolved, into a tenth solution of HF+HClO₄, evaporating the result at the temperature of 110° C. to completely dry the result, introducing the result into 12M HCl solution, heating the result for 30 minutes, and completely dissolving the fifth sample to obtain an eleventh solution (step S50).

In addition, steps S10, S20, S30, S40, and S50 further includes steps S15, S25, S35, S45, and S55 to analyze the contents of a Pb isotopes existing in Pb isotope effluents of the second, fourth, sixth, ninth, and eleventh solution in steps S10, S20, S30, S40, and S50.

In this case, the step 5 (step S50) (and analysis step S55) may be omitted according to occasions. If the step 5 (step S50) is omitted, the artificial contamination source resulting from the industrial activity of a human being may be significantly advantageously specified, and the detail thereof will be described below.

In step 1 to 5 (step S20 to step S50), Pb isotopes are eluted at 5 types of “cation exchange fraction”, “carbonate fraction”, “iron-oxide and manganese hydroxide-fraction”, “organic matters and sulfide fraction”, and “residual fraction” existing at other types (mainly, the type of a silicate). The samples are continuously partially eluted at the above fraction types in the above steps.

For reference, the steps 1 to 5 may be named first step to fifth step or “FI”, “FII”, “FIII”, “FIV”, and “FV”. Hereinafter, according to the occasions, the steps 1 to 5 (or the first step to the fifth step) may be substituted with “FI”, “FII”, “FIII”, “FIV”, and “FV”, respectively, for the description.

4-1. Step 1 (Step S10)

In step 1 (step S10), after preparing the first solution of 1M MgCl₂ (pH=7), the first sample, which had been previously prepared, was introduced into the first solution and stirred for one hour at the normal temperature to obtain the second solution serving as the Pb isotope effluent. In this case, 8 mL of the first solution was prepared, and the quantity of the first solution may be varied depending on the quantity of the first sample. The second solution is a first solution to analyze the Pb isotope.

In this case, the remaining portion of the first sample, which was not dissolved in the first solution, was retrieved as the second sample to be used in the following step.

In this case, if the first sample is previously pulverized in a particle size of 80 meshes to 100 meshes, the first sample may be more easily dissolved in the first solution.

4-2. Step 2 (Step S20)

In step 2 (step S20), after preparing the third solution of 1M CH₃COONa, the pH of the third solution was adjusted to 5 by using HOAc, the second sample retrieved from step S10, which was not dissolved, was introduced and stirred at the normal temperature for five hours to obtain the fourth solution serving as the Pb isotope effluent. In this case, 8 mL of the third solution was prepared, and the quantity of the third solution may be varied depending on the quantity of the second sample. The fourth solution is the second solution to analyze the Pb isotope.

In this case, the remaining portion of the second sample, which was not dissolved in the third solution, was retrieved as the third sample to be used in the following step.

4-3. Step 3 (Step S30)

In step 3 (step S30), after preparing the fifth solution of 0.04M NH₂OH.HCl+25% HOAc, the pH of the fifth solution was adjusted to 2, the third sample retrieved from step S20, which was not dissolved, was introduced and stirred at the temperature of 96° C. for six hours to obtain the sixth solution serving as the Pb isotope effluent. In this case, 20 mL of the fifth solution was prepared, and the quantity of the fifth solution may be varied depending on the quantity of the third sample. The sixth solution is the third solution to analyze the Pb isotope.

In this case, the remaining portion of the third sample, which was not dissolved in the fifth solution, was retrieved as the third fourth to be used in the following step.

4-4. Step 4 (Step S40)

In step 4 (step S40), after preparing the seventh solution of 30% H₂O₂+0.02M HNO₃, the fourth sample retrieved from step S30, which was not dissolved, was introduced, heated at the temperature of 85° C. for five hours and cooled. Then, the eighth solution of 3.2M NH₄OAc+20% HNO₃ was additionally introduced and stirred at the normal temperature for 30 minutes to the ninth solution serving as the Pb isotope effluent (step S40). In this case, 20 mL of the seventh solution was prepared, and the quantity of the seventh solution may be varied depending on the quantity of the first sample. The ninth solution is the third fourth to analyze the Pb isotope.

In this case, the remaining portion of the fourth sample, which was not dissolved in the eighth solution, was retrieved as the fifth fourth to be used in the following step.

4-5. Step 5 (S50)

In step 5 (step S50), after introducing the fifth sample retrieved in step S40, which was not dissolved, into the tenth solution of HF+HClO₄, the result was evaporated at the temperature of 110° C. and completely dried. Next, the result was introduced into a 12M HCl solution and heated for 30 minutes so that the fifth sample was completely dissolved to obtain the eleventh solution serving as the Pb isotope effluent (step S50). Then, 25 mL of the tenth solution was prepared, and the quantity of the tenth solution may be varied depending on the quantity of the fifth sample. The eleventh solution is the fifth solution to analyze the Pb isotope.

The above steps S10 to S50 may further include steps S15, S25, S35, S45, and S55 to analyze the content of Pb isotopes in the second solution, the fourth solution, the sixth solution, the ninth solution, and the eleventh solution obtained from the Pb isotope effluent.

4-6. Step S15 to Step S55

The above steps S15 to S55 are steps to analyze the content of Pb isotopes in the second solution, the fourth solution, the sixth solution, the ninth solution, and the eleventh solution obtained from the Pb isotope effluent.

The content analysis results of the Pb isotopes in the second solution to the eleventh solution are summarized as shown in table 1.

In table 1, Non Asian dust represents an atmospheric floater sample, Asian dust represents a yellow dust sample generated from China, China desert soil represents a desert soil sample, and FI-3 to FV-19 represent sample numbers. In addition, ²⁰⁶Pb/²⁰⁴Pb represents the ratio between contents of Pb isotopes.

TABLE 1

| | Sample | ²⁰⁶Pb/²⁰⁴Pb | ²⁰⁷Pb/²⁰⁴Pb | ²⁰⁸Pb/²⁰⁴Pb | ²⁰⁸Pb/²⁰⁶Pb | ²⁰⁶Pb/²⁰⁷Pb |
|---|---|---|---|---|---|---|
| Non | FI-3 | 18.005 | 15.624 | 38.14 | 2.1183 | 1.1524 |
| Asian | FI-4 | 17.9053 | 15.6147 | 37.9785 | 2.1181 | 1.1475 |
| dust | | | | | | |
| Asian | FI-6 | 17.8099 | 15.6237 | 37.8062 | 2.1227 | 1.14 |
| dust | FI-7 | 18.0598 | 15.6906 | 38.1594 | 2.1127 | 1.1511 |
| | FI-8 | 18.1813 | 15.6108 | 38.2387 | 2.1031 | 1.1647 |
| | FI-9 | 17.944 | 15.592 | 37.9326 | 2.1139 | 1.1508 |
| | FI-10 | 18.3737 | 15.8191 | 38.4711 | 2.0942 | 1.1617 |
| Non | FII-3 | 17.9922 | 15.6096 | 38.129 | 2.1191 | 1.1526 |
| Asian | FII-4 | 17.9123 | 15.5927 | 37.9551 | 2.1188 | 1.1488 |
| dust | | | | | | |
| Asian | FII-5 | 18.1406 | 15.5942 | 38.2717 | 2.1097 | 1.1633 |
| dust | FII-6 | 17.7331 | 15.5749 | 37.6753 | 2.1247 | 1.1386 |
| | FII-7 | 17.8952 | 15.594 | 37.9049 | 2.1181 | 1.1476 |
| | FII-8 | 18.1819 | 15.6084 | 38.2446 | 2.1035 | 1.1648 |
| | FII-9 | 17.9708 | 15.5916 | 37.9851 | 2.1137 | 1.1526 |
| | FII-10 | 18.104 | 15.6083 | 38.1418 | 2.1068 | 1.1599 |
| Chinese | FII-13 | 18.7521 | 15.6598 | 38.7409 | 2.0658 | 1.1975 |
| desert | FII-14 | 18.8589 | 15.6896 | 38.8071 | 2.0579 | 1.2019 |
| soil | FII-15 | 18.7139 | 15.6666 | 38.7873 | 2.0725 | 1.1944 |
| | FII-16 | 18.3951 | 15.6635 | 38.4299 | 2.0899 | 1.1744 |
| | FII-17 | 18.6667 | 15.6643 | 38.784 | 2.0777 | 1.1918 |
| | FII-18 | 18.6784 | 15.6744 | 38.8414 | 2.0795 | 1.1916 |
| | FII-19 | 18.682 | 15.6669 | 38.8053 | 2.0772 | 1.1924 |
| Non | FIII-3 | 18.1144 | 15.6277 | 38.2802 | 2.1132 | 1.1589 |
| Asian | FIII-4 | 18.0006 | 15.6161 | 38.1044 | 2.1169 | 1.1526 |
| dust | | | | | | |
| Asian | FIII-5 | 18.2125 | 15.6069 | 38.3477 | 2.1056 | 1.1669 |
| dust | FIII-6 | 17.8385 | 15.5962 | 37.8586 | 2.1223 | 1.1437 |
| | FIII-7 | 17.937 | 15.5977 | 37.9703 | 2.1166 | 1.15 |
| | FIII-8 | 18.2003 | 15.6114 | 38.2649 | 2.1032 | 1.1659 |
| | FIII-9 | 18.0253 | 15.6145 | 38.09 | 2.1132 | 1.1544 |
| | FIII-10 | 18.1332 | 15.6176 | 38.2168 | 2.1077 | 1.1611 |
| Chinese | FIII-13 | 18.7018 | 15.6641 | 38.7872 | 2.074 | 1.1939 |
| desert | FIII-14 | 18.7953 | 15.6709 | 38.7953 | 2.064 | 1.1994 |
| soil | FIII-15 | 18.7405 | 15.6783 | 38.8781 | 2.0745 | 1.1953 |
| | FIII-16 | 18.5595 | 15.6489 | 38.6383 | 2.0819 | 1.186 |
| | FIII-17 | 18.6825 | 15.6591 | 38.8036 | 2.0771 | 1.1931 |
| | FIII-18 | 18.6722 | 15.6561 | 38.8068 | 2.0789 | 1.1923 |
| | FIII-19 | 18.6636 | 15.6476 | 38.7767 | 2.0777 | 1.1927 |
| Non | FIV-3 | 18.1035 | 15.667 | 38.3937 | 2.121 | 1.1554 |
| Asian | FIV-4 | 18.0558 | 15.6439 | 38.2167 | 2.1166 | 1.1542 |
| dust | | | | | | |
| Asian | FIV-5 | 18.2776 | 15.653 | 38.4691 | 2.1048 | 1.1677 |
| dust | FIV-6 | 17.8995 | 15.6156 | 37.9762 | 2.1215 | 1.1463 |
| | FIV-7 | 18.0026 | 15.6355 | 38.09 | 2.1154 | 1.1516 |
| | FIV-8 | 18.1876 | 15.6094 | 38.2573 | 2.1033 | 1.1652 |
| | FIV-9 | 18.0216 | 15.6081 | 38.0736 | 2.1127 | 1.1546 |
| | FIV-10 | 18.1251 | 15.6039 | 38.1902 | 2.1075 | 1.1613 |
| Chinese | FIV-13 | 18.8098 | 15.6843 | 38.8092 | 2.0631 | 1.1992 |
| desert | FIV-14 | 18.9519 | 15.6996 | 38.9793 | 2.0566 | 1.2072 |
| soil | FIV-15 | 18.9805 | 15.718 | 39.0789 | 2.0589 | 1.2076 |
| | FIV-16 | 18.7318 | 15.6999 | 38.7755 | 2.07 | 1.1931 |
| | FIV-17 | 18.8619 | 15.6788 | 38.9263 | 2.0637 | 1.203 |
| | FIV-18 | 18.724 | 15.6637 | 38.8666 | 2.0757 | 1.1954 |
| | FIV-19 | 18.7499 | 15.6639 | 38.8509 | 2.0721 | 1.197 |
| Non | FV-3 | 18.4744 | 15.8364 | 39.0675 | 2.1142 | 1.1665 |
| Asian | FV-4 | 18.3559 | 15.7595 | 38.8148 | 2.1145 | 1.1648 |
| dust | | | | | | |
| Asian | FV-6 | 18.4347 | 15.7268 | 38.728 | 2.1004 | 1.1722 |
| dust | FV-7 | 18.4987 | 15.7766 | 38.8642 | 2.1013 | 1.1726 |
| | FV-8 | 18.5974 | 15.7865 | 38.9217 | 2.0929 | 1.1781 |
| | FV-9 | 18.5234 | 15.8115 | 38.8924 | 2.0996 | 1.1714 |
| | FV-10 | 18.505 | 15.702 | 38.7972 | 2.0967 | 1.1785 |
| Chinese | FV-13 | 17.9873 | 15.5526 | 38.0692 | 2.1163 | 1.1562 |
| desert | FV-14 | 18.8506 | 15.6939 | 38.9836 | 2.068 | 1.2011 |
| soil | FV-15 | 18.6755 | 15.6907 | 38.9539 | 2.0855 | 1.1904 |
| | FV-16 | 18.6629 | 15.6948 | 38.8403 | 2.0808 | 1.1892 |
| | FV-17 | 18.9352 | 15.7473 | 39.3249 | 2.0763 | 1.2028 |
| | FV-18 | 18.965 | 15.7742 | 39.3481 | 2.0748 | 1.2022 |
| | FV-19 | 18.7788 | 15.7211 | 39.1685 | 2.0857 | 1.1945 |

Next, the values shown in table 1, which have been statistically processed, are shown in table 2. The values shown in table 2 represent each numerical value range shown in table 1, an average value of the numeric value range, and a standard deviation (s.d.).

TABLE 2

| | Sample | [symbol] | [symbol] | [symbol] | [symbol] | [symbol] |
|---|---|---|---|---|---|---|
| Non Asian dust [symbol] | range | [symbol] | [symbol] | [symbol] | [symbol] | [symbol] |
| Asian dust [symbol] | average [symbol] | [symbol] | [symbol] | [symbol] | [symbol] | [symbol] |
| | range | [symbol] | [symbol] | [symbol] | [symbol] | [symbol] |
| | average [symbol] | [symbol] | [symbol] | [symbol] | [symbol] | [symbol] |
| Non Asian dust [symbol] | range | [symbol] | [symbol] | [symbol] | [symbol] | [symbol] |
| Asian dust [symbol] | average [symbol] | [symbol] | [symbol] | [symbol] | [symbol] | [symbol] |
| Chinese desert soil [symbol] | range | [symbol] | [symbol] | [symbol] | [symbol] | [symbol] |
| | average [symbol] | [symbol] | [symbol] | [symbol] | [symbol] | [symbol] |
| | range | [symbol] | [symbol] | [symbol] | [symbol] | [symbol] |
| | average [symbol] | [symbol] | [symbol] | [symbol] | [symbol] | [symbol] |
| Non Asian dust [symbol] | range | [symbol] | [symbol] | [symbol] | [symbol] | [symbol] |
| Asian dust [symbol] | average [symbol] | [symbol] | [symbol] | [symbol] | [symbol] | [symbol] |
| Chinese desert soil [symbol] | range | [symbol] | [symbol] | [symbol] | [symbol] | [symbol] |
| | average [symbol] | [symbol] | [symbol] | [symbol] | [symbol] | [symbol] |
| | range | [symbol] | [symbol] | [symbol] | [symbol] | [symbol] |
| | average [symbol] | [symbol] | [symbol] | [symbol] | [symbol] | [symbol] |
| Non Asian dust [symbol] | range | [symbol] | [symbol] | [symbol] | [symbol] | [symbol] |
| Asian dust [symbol] | average [symbol] | [symbol] | [symbol] | [symbol] | [symbol] | [symbol] |
| Chinese desert soil [symbol] | range | [symbol] | [symbol] | [symbol] | [symbol] | [symbol] |
| | average [symbol] | [symbol] | [symbol] | [symbol] | [symbol] | [symbol] |
| | range | [symbol] | [symbol] | [symbol] | [symbol] | [symbol] |
| | average [symbol] | [symbol] | [symbol] | [symbol] | [symbol] | [symbol] |
| Non Asian dust [symbol] | range | [symbol] | [symbol] | [symbol] | [symbol] | [symbol] |
| Asian dust [symbol] | average [symbol] | [symbol] | [symbol] | [symbol] | [symbol] | [symbol] |
| Chinese desert soil [symbol] | range | [symbol] | [symbol] | [symbol] | [symbol] | [symbol] |
| | average [symbol] | [symbol] | [symbol] | [symbol] | [symbol] | [symbol] |
| | range | [symbol] | [symbol] | [symbol] | [symbol] | [symbol] |
| | average [symbol] | [symbol] | [symbol] | [symbol] | [symbol] | [symbol] |

[symbol]

indicates data missing or illegible when filed

### Embodiment

Hereinafter, the constitution and the operation of the present invention will be described in more detail according to the exemplary embodiment of the present invention. The exemplary embodiment is provided only for the illustrative purpose, and the present invention is not limited thereto.

Since other advantages and other characteristics that are not described herein can be sufficiently and technically comprehended by those skilled in the art, the details thereof will be omitted in order to avoid redundancy.

1. Sample Preparation

As described above, Asian dust was selected as a representative sample moving between nations.

Since the Asian dust is generated from Chinese continental inner zones and moves beyond Korea through a Chinese continent, the Asian dust is a desirable sample to investigate causes regarding whether the Pb contamination source contained in the Asian dust refers to a generation point of the Asian dust, an artificial contamination source is added to the Asian dust due to the industrial activity in the Chinese content during the movement of the Asian dust, and the Asian dust is exposed to even an artificial Pb contamination source in Korea after moving to Korea.

For the comparison with Asian dust, as shown in table 1, non Asian dust was collected together with the Asian dust.

In detail, 0.6 g of an Asian dust sample and 0.4 g of a non Asian dust sample were collected.

Since the Asian dust sample and the non Asian dust sample are fine dust, the Asian dust sample and the non Asian dust sample do not require a pulverization process. However, it is preferred that samples having the form of other deposits are previously pulverized in the particle size of 80 meshes to 100 meshes.

2. Sequential Extraction Scheme

According to the sequential extraction scheme, the analysis results obtained by extracting Pb isotopes from the sample through step 1 (step S10) to step 5 (step S50) are shown in table 1, and the values shown in table 1 are summarized in table 2.

3. Content Analysis Result of Pb Isotope

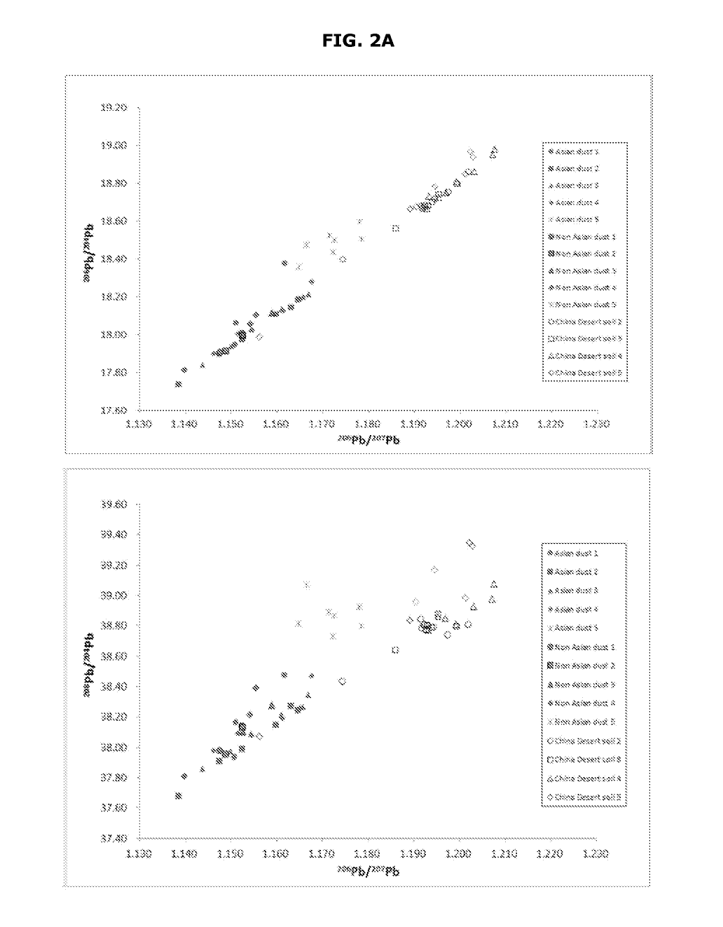

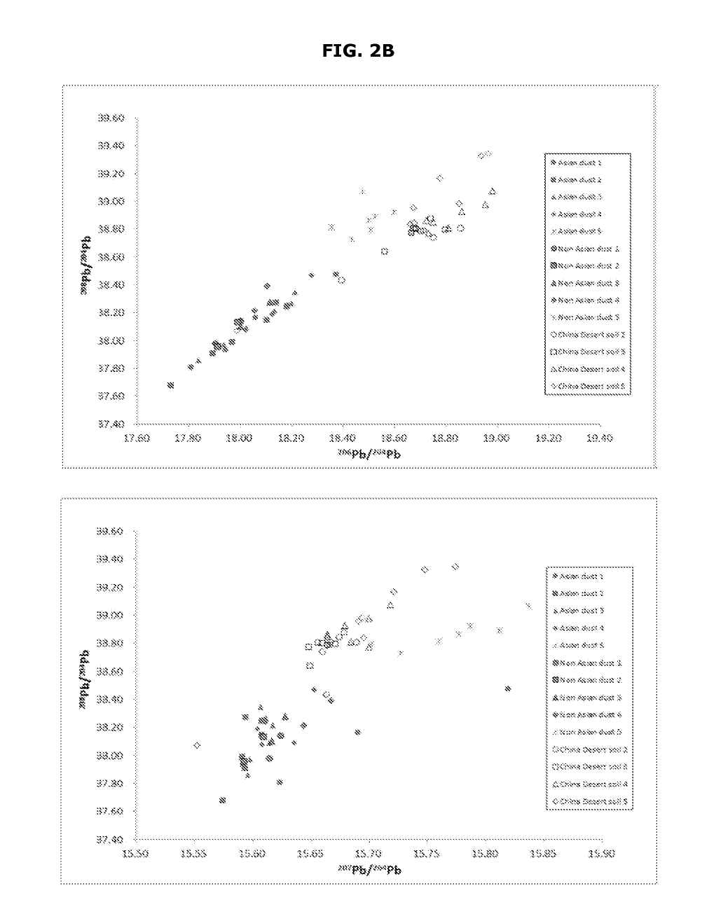

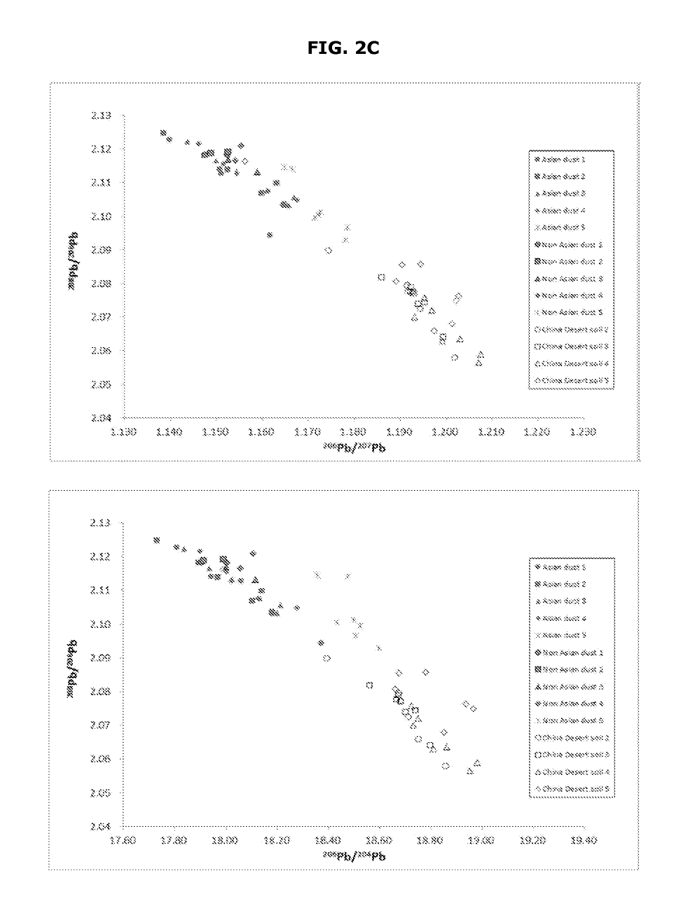

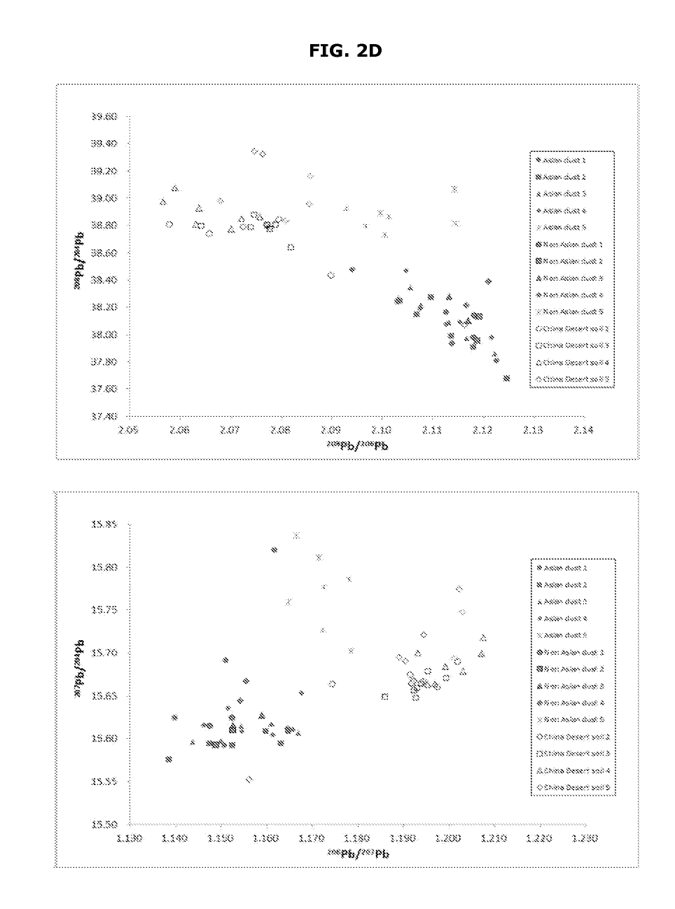

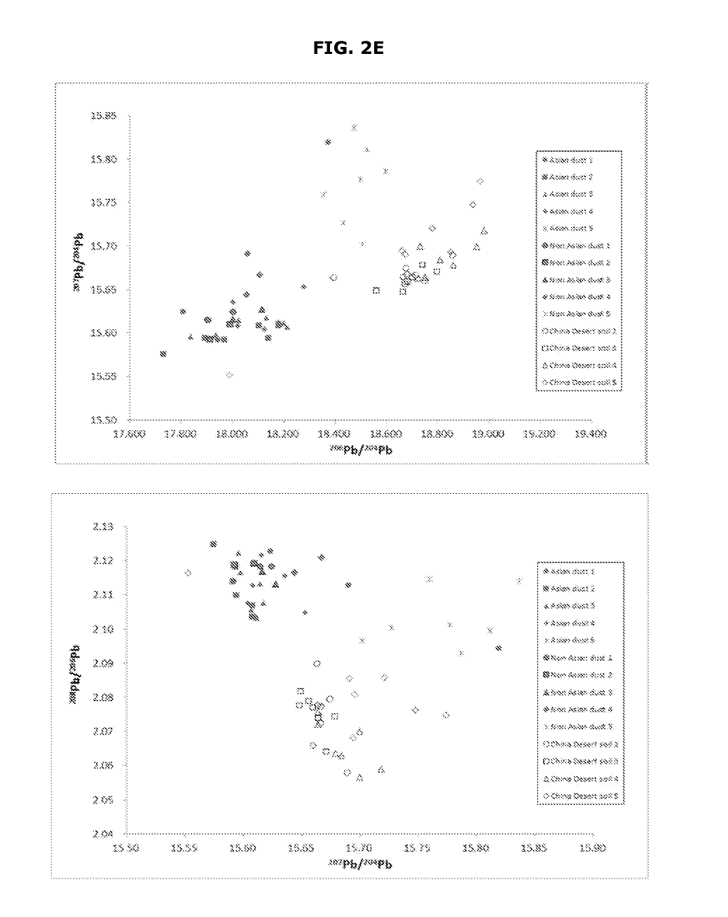

FIGS. 2A to 2E are graphs showing analysis results according to the method of resolving the heavy metal contamination source based on the sequential extraction scheme and the isotope analysis scheme according to the exemplary embodiment of the present invention.

Hereinafter, the analysis result will be described with reference to the information shown in table 1 and 2, and FIGS. 2A to 2E.

First, regarding the content ratios of ²⁰⁶Pb/²⁰⁷Pb isotopes, the Asian dust and the non Asian dust have the approximate content ratios of ²⁰⁶Pb/²⁰⁷Pb isotopes from “cation exchange fraction (obtained in step FI)” to “organic matters and sulfide fraction (obtained in step FIV)”

In detail, the average value in the cation exchange fraction (obtained in step FI) was 1.1537±0.0098 (1.1400˜1.1647), the average value in the carbonate fraction (obtained in step FII) was 1.1545±0.0102 (1.1386˜1.1648), the average value in the iron-oxide and manganese hydroxide-fraction (obtained in step FIII) was 1.1570±0.0092 (1.1437˜1.1669), and the average value in the organic matters and sulfide fraction (obtained in step FIV) was 1.1578±0.0083 (1.1463˜1.1677), which represent very approximate values.

However, in the case of “residual fraction” (obtained in FV), the average value is 1.1746±0.0034 (1.1714˜1.1785), which represents the significant difference from other values.

The above results refer to that the Pb isotopes in steps FI to FIV constituting the sequential extraction have the same origin or the same beginning, but the Pb isotope in step FV has an origin different from those of the Pb isotopes in steps FI to FIV.

Meanwhile, the non Asian dust shows the result similar to that of the Asian dust.

In detail, in the case of the non Asian dust, the average value in step FI is 1.1500±0.0035 (1.1475˜1.1524), the average value in step FII is 1.1507±0.0027 (1.1488˜1.1526), the average value in step FIII is 1.1558±0.0045 (1.1526˜1.1589), and the average value in step FIV is 1.1548±0.0008 (1.1542˜1.1554), which represent approximate results in the above steps.

The above values are significantly approximate to the values of the Asian dust. However, the average value in step FV is represented as 1.1657±0.0012 (1.1648˜1.1665), which represents a significant difference from the values in other steps.

The above difference refers to that the Pb isotopes existing in Asian dust and non Asian dust have the same Pb origins as those of Pb isotopes existing at the type of “cation exchange fraction (obtained in step FI)”, “carbonate fraction (obtained in step FII)”, “Fe—Mn hydroxide-fraction (obtained in step FIII)”, and “organic matters and sulfide fraction (obtained in step FIV)”, but have origins different from those of the Pb isotopes existing in residual fraction (obtained in step FV) such as other silicates.

Meanwhile, in the case of the China desert soil, the average value of the content ratio of ²⁰⁶Pb/²⁰⁷Pb obtained in step FII is 1.1920±0.0086 (1.1744˜1.2019), the average value of the content ratio of ²⁰⁶Pb/²⁰⁷Pb obtained in step FII is 1932±0.0040 (1.1860˜1.1994), the average value of the content ratio of ²⁰⁶Pb/²⁰⁷Pb obtained in step FIV is 1.2004±0.0057 (1.1931˜1.2076), and the average value of the content ratio of ²⁰⁶Pb/²⁰⁷Pb obtained in step FV is 1.909±0.0163 (1.1562˜1.2028)

That is to say, in the case of a China desert soil, the content ratios of ²⁰⁶Pb/²⁰⁷Pb isotopes obtained from step FII to step FV, which is the final step, represent substantially approximate values regardless of the sequential extraction step.

The above result refers to that the substantially approximate content ratios of ²⁰⁶Pb/²⁰⁷Pb isotopes are represented regardless of the type of Pb isotopes contained in the China desert soil, that is, the types of minerals such as, “carbonate fraction”, “Fe—Mn hydroxide-fraction”, “organic matters and sulfide fraction”, or “silicates. This refers to that the Pb isotopes have the same origin regardless of the physical and chemical types of Pb isotopes.

In other words, in the case of the China desert soil, all origins of heavy metal such as Pb existing in the China desert soil are the same. To the contrary, the Asian dust and the non Asian dust represent the same origin of Pb heavy metal contained at the type of “cation exchange fraction”, “carbonate fraction”, “Fe—Mn hydroxide-fraction”, “organic matters and sulfide fraction”, but the Pb isotope existing at the type of “residual fraction” such as silicates represents a different Pb origin.

Therefore, the Asian dust and the non Asian dust represent at least two heavy metal contamination sources including a Pb isotope.

However, in step FV, although the content ratios of ²⁰⁶Pb/²⁰⁷Pb isotopes represent the same value in the Asian dust, the non Asian dust, and the China desert soil, content ratios of ²⁰⁶Pb/²⁰⁷Pb isotopes, which are actually measured, represent different values, especially, a value significantly lower than the content ratio of ²⁰⁶Pb/²⁰⁷Pb isotope of the China desert soil.

It is estimated that the content ratios of ²⁰⁶Pb/²⁰⁷Pb isotopes may be reduced because heavy metal elements such as Pb are affected by external contaminants in the Asian dust and the non Asian dust.

The result is identically represented in the content ratio of a ²⁰⁶Pb/²⁰⁴Pb isotope, which supports that the above result is right.

In detail, the Asian dust and the non Asian dust represent a significantly approximate value in the content ratio of a ²⁰⁶Pb/²⁰⁴Pb isotope in step FIV from step FI, which makes a significant difference from the content ratio of an isotope in step FV.

To the contrary, the China desert soil represents significantly approximate content ratios of the isotopes in step FV from step FII.

The above result is identically represented in the content ratio of a ²⁰⁶Pb/²⁰⁷Pb isotope.

However, in step FV, the content ratio of a ²⁰⁶Pb/²⁰⁴Pb isotope represents a value significantly approximate to the average value of 18.5118±0.0584 (18.4347˜18.5974) in the Asian dust, the average value of 18.4152±0.0838 (18.3559˜18.4744) in the non Asian dust, and the average value of 18.6936±0.3327 (17.9873˜18.9650) in a China desert soil.

Therefore, in step FV, when comparing the content ratios of ²⁰⁶Pb/²⁰⁴Pb isotopes with each other, the same origin is represented in the Asian dust, the non Asian dust, and the desert soil.

However, the above description shows that the average value in the content ratios of ²⁰⁶Pb/²⁰⁴Pb isotopes in step FV is different from the average value in the content ratios of ²⁰⁶Pb/²⁰⁷Pb isotopes in step FV.

However, in order to investigate the origins of the Asian dust, the non Asian dust, and the China desert soil in step FV, the use of the average value in the content ratio of the ²⁰⁶Pb/²⁰⁷Pb isotope is more exact than the use of the average value in the content ratio of ²⁰⁶Pb/²⁰⁴Pb isotope.

Meanwhile, the average value in the content ratio of a ²⁰⁸Pb/²⁰⁶Pb isotope is different from the average value in the content ratio of a ²⁰⁶Pb/²⁰⁷Pb isotope and the average value in the content ratio of a ²⁰⁶Pb/²⁰⁴Pb isotope.

In detail, the average value in the content ratio of a ²⁰⁸Pb/²⁰⁶Pb isotope represents the significantly approximate value in the Asian dust and the non Asian dust in step FI to step FIV, but represents a slight lowered value. In the case of the Asian dust, the average value in the content ratio of the ²⁰⁸Pb/²⁰⁶Pb isotope represents a significantly lowered value.

However, the China desert soil represents the significantly approximate average value in the content ratio of a ²⁰⁸Pb/²⁰⁶Pb isotope in step FII to step FIV, and represents the slightly increased average value in the content ratio of a ²⁰⁸Pb/²⁰⁶Pb isotope in step FV.

The result is opposite to a result in the average value in the content ratio of the ²⁰⁶Pb/²⁰⁷Pb isotope, which is caused by the external contamination of the Pb heavy metal element.

Meanwhile, regarding the average value in the content ratio of the ²⁰⁸Pb/²⁰⁴Pb isotope, all target samples of the Asian dust, the non Asian dust, and the China desert soil represent the different average values in the content ratio of the ²⁰⁸Pb/²⁰⁴Pb isotope in step FI to step FV, and then represent the slightly increased average value in the content ratio of the ²⁰⁸Pb/²⁰⁴Pb isotope in the following step.

However, in step FV, the average values in the content ratio of the ²⁰⁸Pb/²⁰⁴Pb isotope represent significantly approximate values in the Asian dust, the non Asian dust, and the China desert soil.

Therefore, it may be determined if the origins of the Pb contamination sources are the same by comparing with the average values of ²⁰⁸Pb/²⁰⁴Pb isotopes in step FV, which is the final step in the sequential extraction scheme.

To this end, after removing heavy metal elements causing external contamination by performing physical and chemical pre-treatment required in step FI to step FIV, the average value in the content ratio of the ²⁰⁸Pb/²⁰⁴Pb isotope must be measured.

Meanwhile, regarding the average value in the content ratio of the ²⁰⁷Pb/²⁰⁴Pb isotope, the average value in the content ratio of the ²⁰⁷Pb/²⁰⁴Pb isotope in the Asian dust, the non Asian dust, and the China desert soil does not represent a predetermined tendency in all sequential extraction steps of step FI to step FV. Accordingly, the use of the content ratio of the above isotope is not desirable to investigate the origin of the contamination source.

Particularly, it is not preferred that the average value in the content ratio of the ²⁰⁷Pb/²⁰⁴Pb isotope is used in the Asian dust and the non Asian dust representing a significant high contamination possibility.

However, the China desert soil represents the same average value in the content ratio of the ²⁰⁷Pb/²⁰⁴Pb isotope in steps FII, FIII, FIV and FV.

As a result, it is most preferred that the content ratio of a ²⁰⁶Pb/²⁰⁴Pb isotope and the content ratio of a ²⁰⁶Pb/²⁰⁷Pb isotope are used in order to investigate the contamination source of heavy metal elements such as Pb.

The correlation in the average value in the content ratio of the isotopes is summarized through the graphs shown in FIGS. 2A to 2E.

As shown in FIGS. 2A to 2E, since the correlation of ²⁰⁶Pb/²⁰⁴Pb vs. ²⁰⁶Pb/²⁰⁷Pb represents a positive linear relationship (graph located in the upper side of FIG. 2A), the analysis is easy. In addition, the correlation of ²⁰⁸Pb/²⁰⁴Pb vs. ²⁰⁶Pb/²⁰⁷Pb and the correlation of ²⁰⁸Pb/²⁰⁴Pb vs. ²⁰⁶Pb/²⁰⁴Pb represent a superior linear relationship.

In addition to the correlation representing the positive linear relationship, the graphs representing the correlation of ²⁰⁸Pb/²⁰⁶Pb vs. ²⁰⁶Pb/²⁰⁷Pb and ²⁰⁸Pb/²⁰⁶Pb vs. ²⁰⁶Pb/²⁰⁴Pb represent a negative linear relationship.

Although all correlation may be used to investigate the origin of the heavy metal including a Pb isotope, it is most preferred that the correlation of ²⁰⁶Pb/²⁰⁴Pb vs. ²⁰⁶Pb/²⁰⁷Pb representing the most clear linear relationship among the above correlation is used.

To the contrary, since the correlation of ²⁰⁸Pb/²⁰⁴Pb vs. ²⁰⁷Pb/²⁰⁴Pb, ²⁰⁸Pb/²⁰⁴Pb vs. ²⁰⁸Pb/²⁰⁶Pb, ²⁰⁷Pb/²⁰⁴Pb vs. ²⁰⁶Pb/²⁰⁷Pb, ²⁰⁷Pb/²⁰⁴Pb vs. ²⁰⁶Pb/²⁰⁴Pb and ²⁰⁸Pb/²⁰⁶Pb vs. ²⁰⁷Pb/²⁰⁴Pb are not clear, the correlation of the ²⁰⁸Pb/²⁰⁴Pb vs. ²⁰⁷Pb/²⁰⁴Pb, ²⁰⁸Pb/²⁰⁴Pb vs. ²⁰⁸Pb/²⁰⁶Pb, ²⁰⁷Pb/²⁰⁴Pb vs. ²⁰⁶Pb/²⁰⁷Pb, ²⁰⁷Pb/²⁰⁴Pb vs. ²⁰⁶Pb/²⁰⁴Pb and ²⁰⁸Pb/²⁰⁶Pb vs. ²⁰⁷Pb/²⁰⁴Pb are not preferred to investigate the origins of the Pb contamination source. Accordingly, it is preferred that the correlation of the ²⁰⁸Pb/²⁰⁴Pb vs. ²⁰⁷Pb/²⁰⁴Pb, ²⁰⁸Pb/²⁰⁴Pb vs. ²⁰⁸Pb/²⁰⁶Pb, ²⁰⁷Pb/²⁰⁴Pb vs. ²⁰⁶Pb/²⁰⁷Pb, ²⁰⁷Pb/²⁰⁴Pb vs. ²⁰⁶Pb/²⁰⁴Pb and ²⁰⁸Pb/²⁰⁶Pb vs. ²⁰⁷Pb/²⁰⁴Pb are not used.

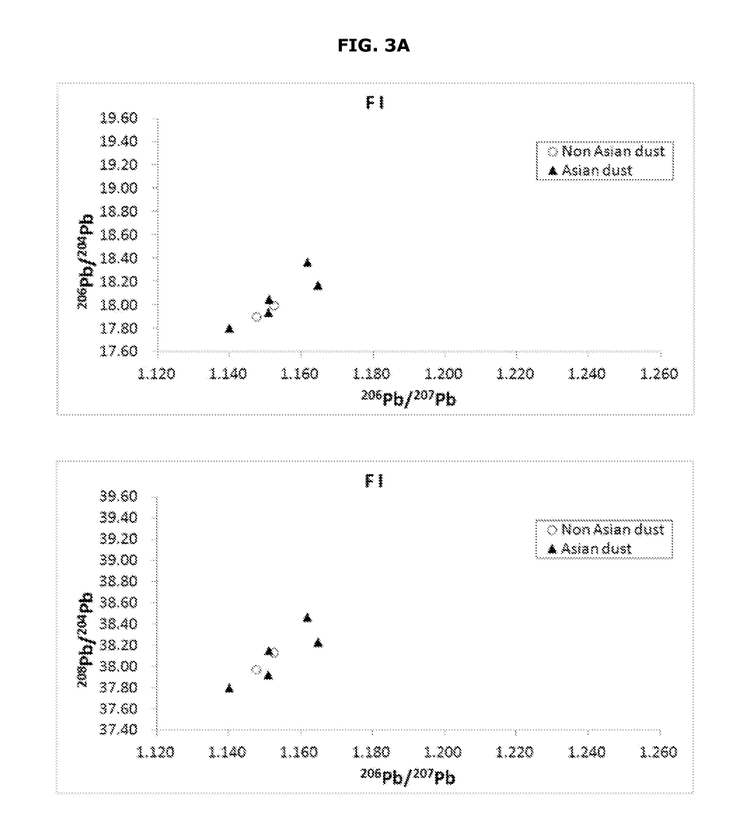

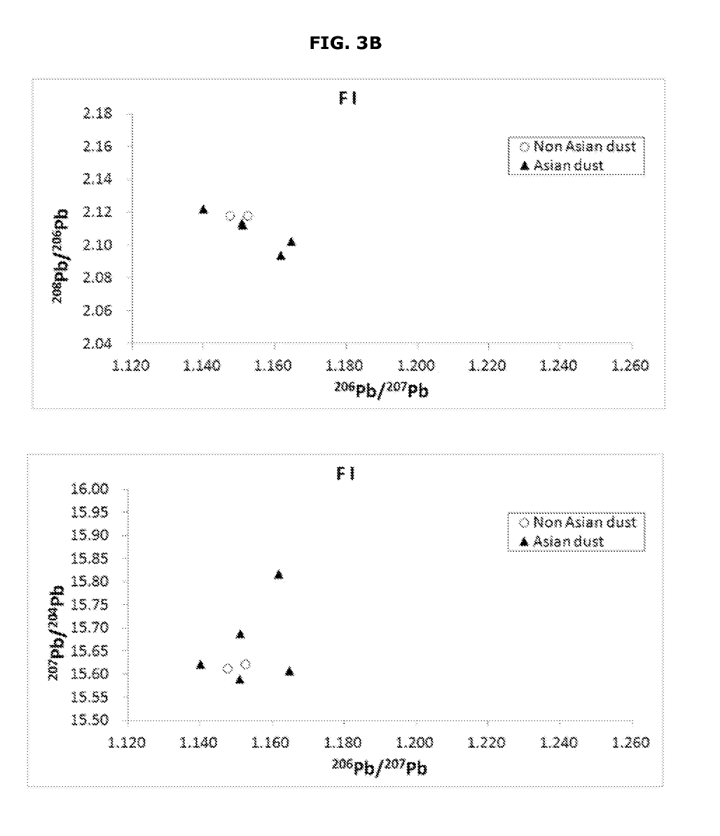

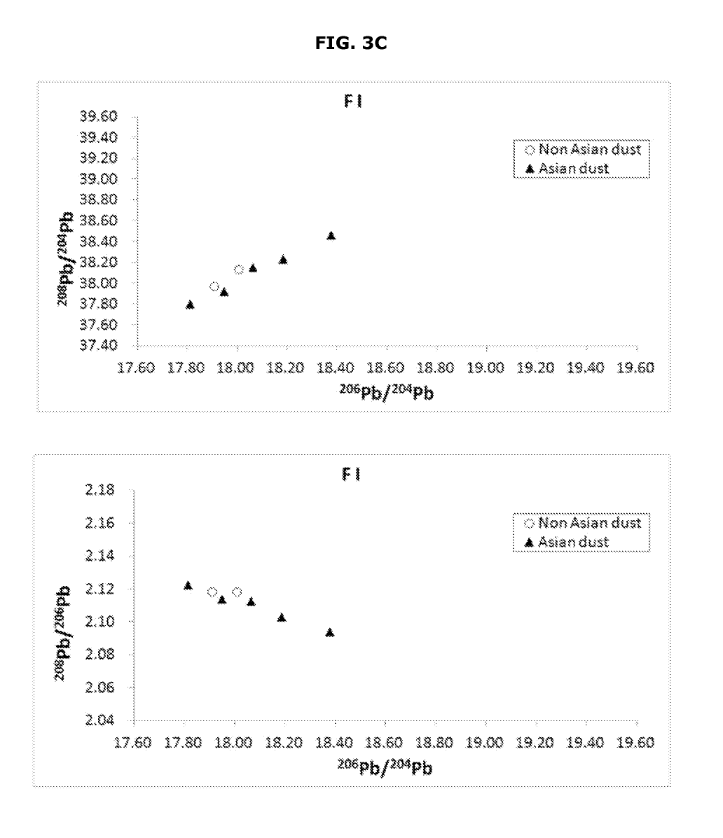

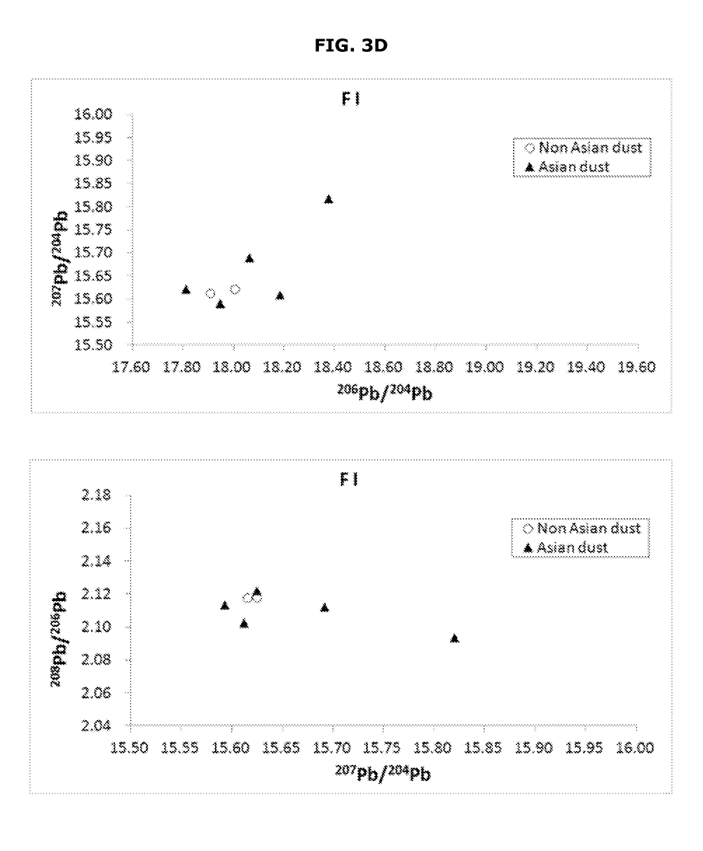

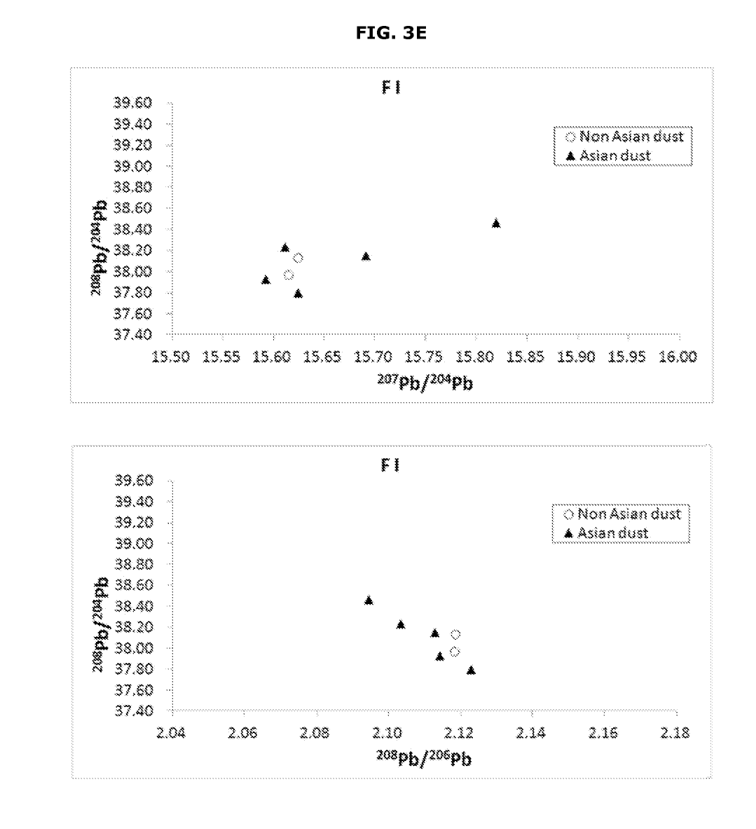

Hereinafter, the extraction characteristic of the Pb isotope in each of steps FI to FV related to the content ratio of the Pb isotope obtained through the sequential extraction scheme will be described with reference to FIGS. 3A to 3E to FIGS. 8A to 8E.

FIGS. 3A to 3E are graphs showing analysis results according to the first step in the method of resolving the heavy metal contamination source based on the sequential extraction scheme and the isotope analysis scheme according to the exemplary embodiment of the present invention.

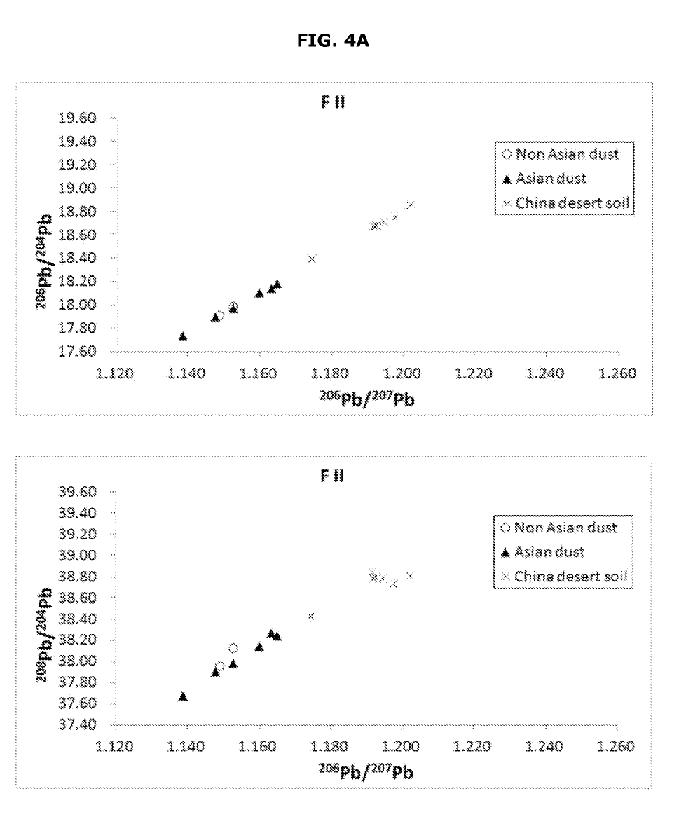

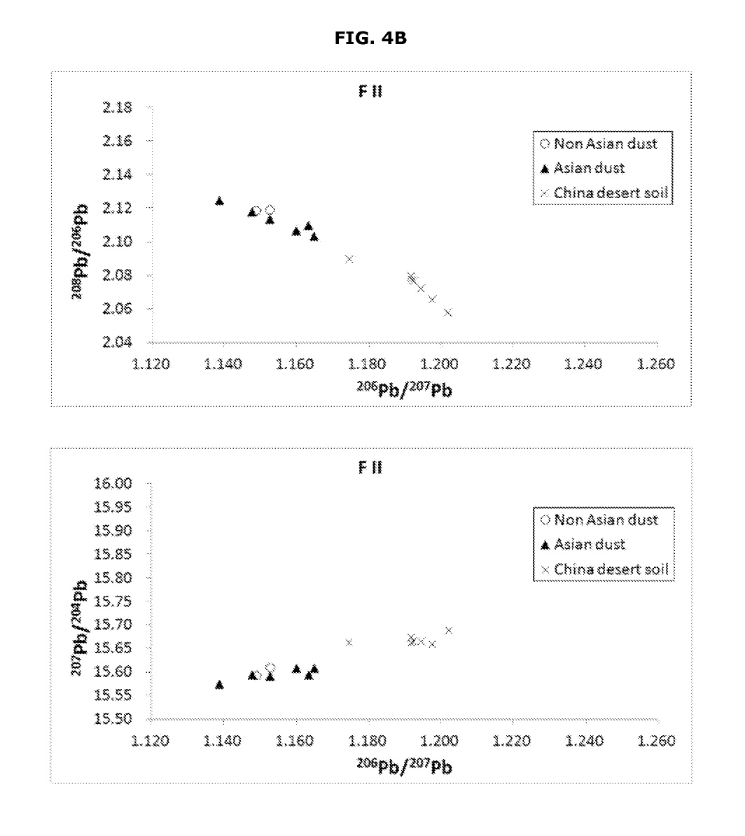

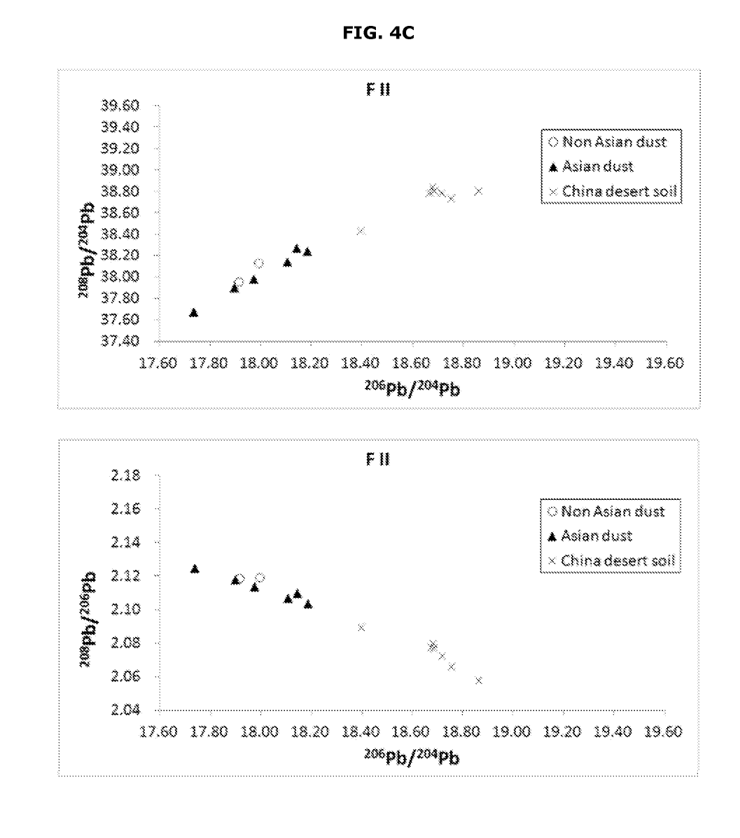

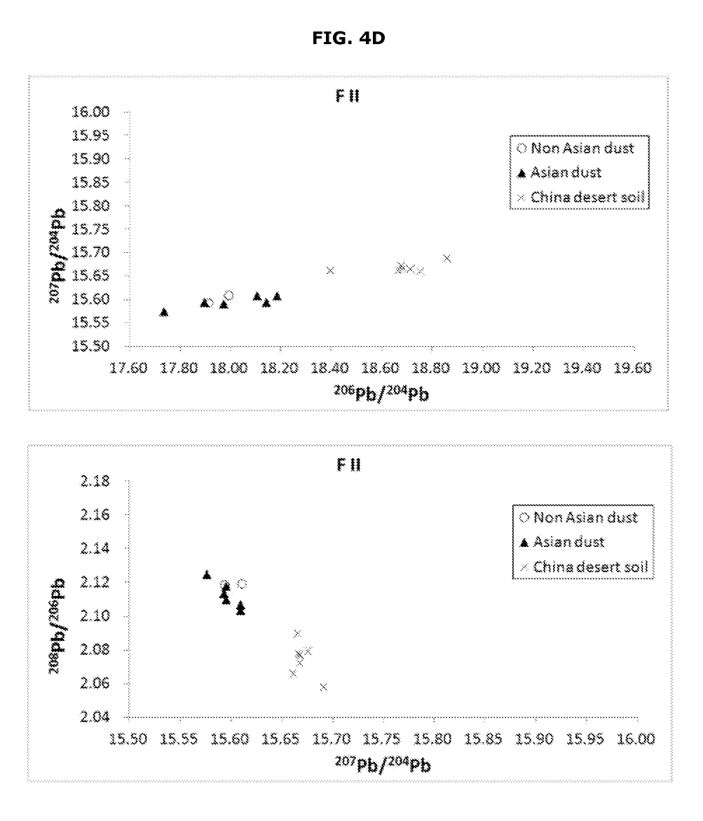

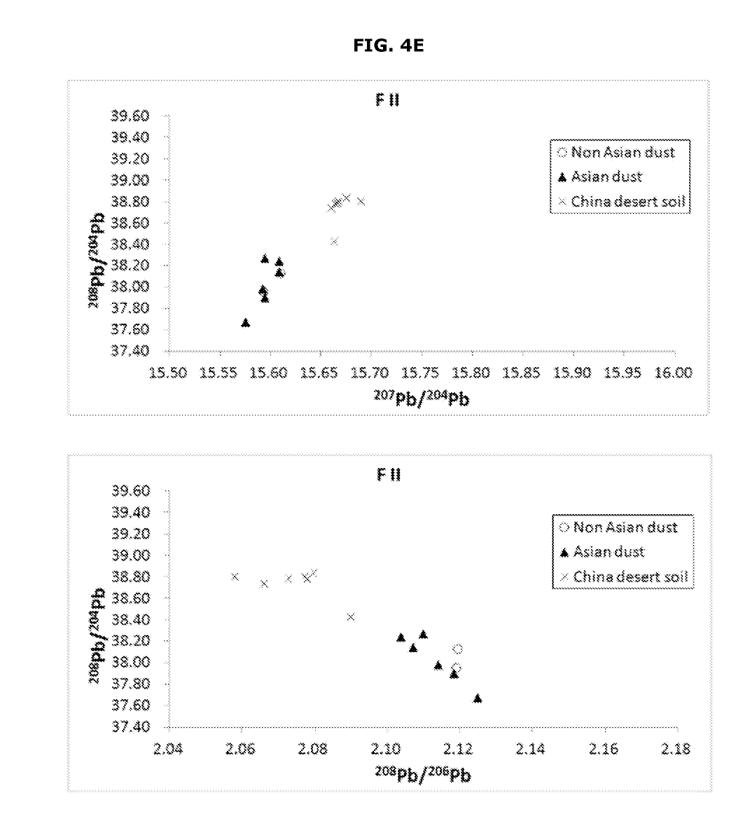

FIGS. 4A to 4E are graphs showing analysis results according to the second step in the method of resolving the heavy metal contamination source based on the sequential extraction scheme and the isotope analysis scheme according to the exemplary embodiment of the present invention.

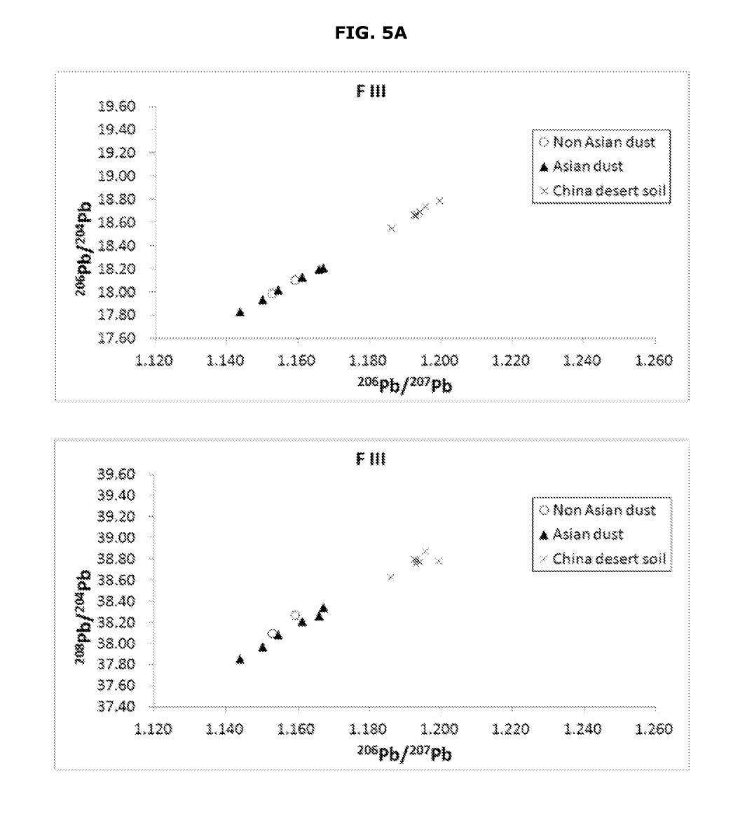

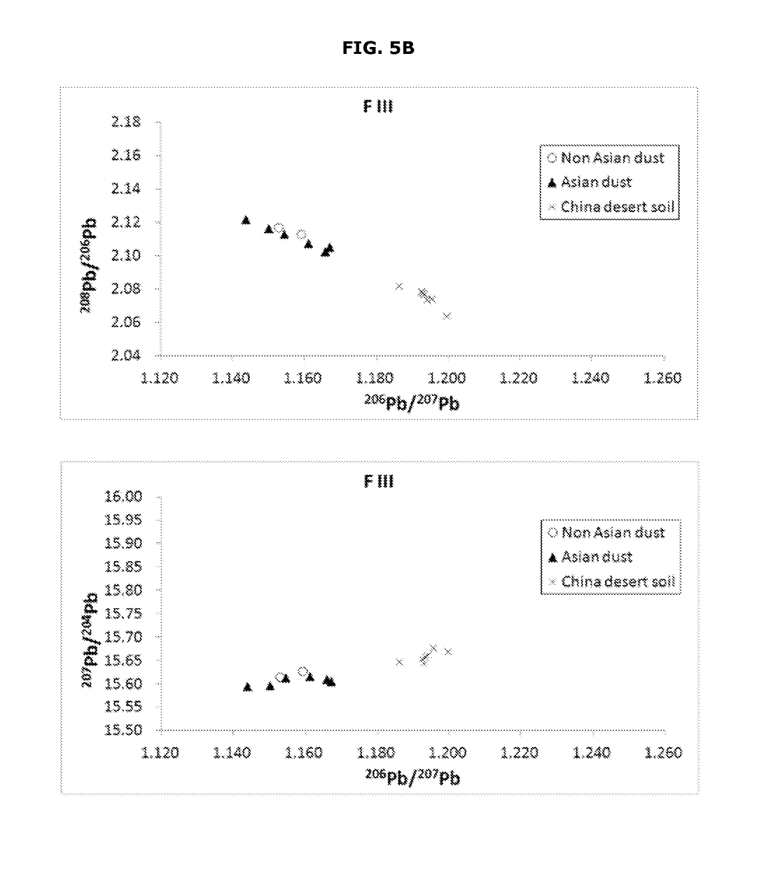

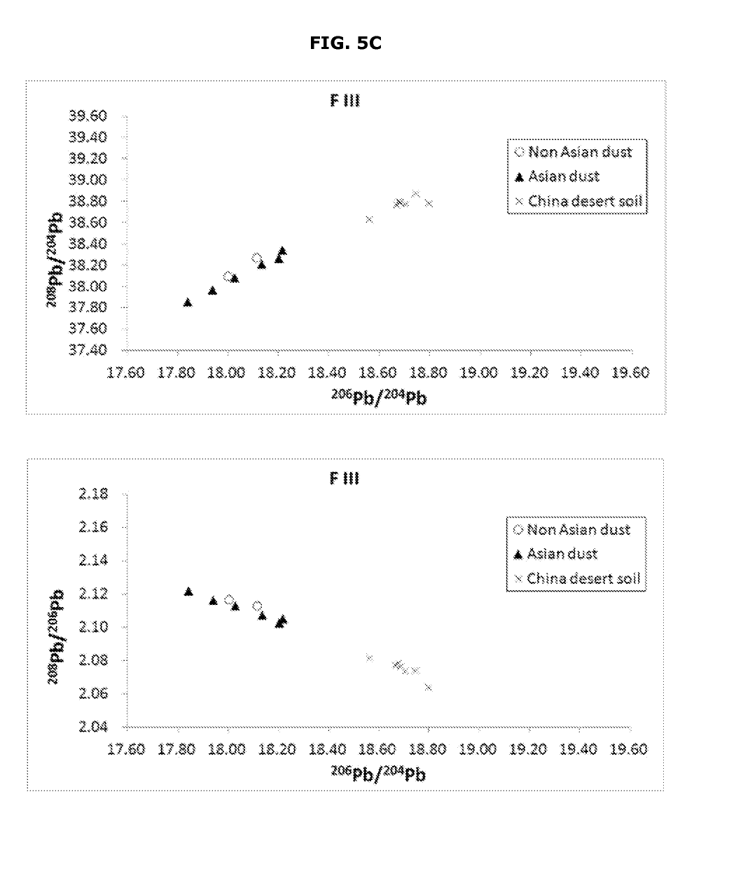

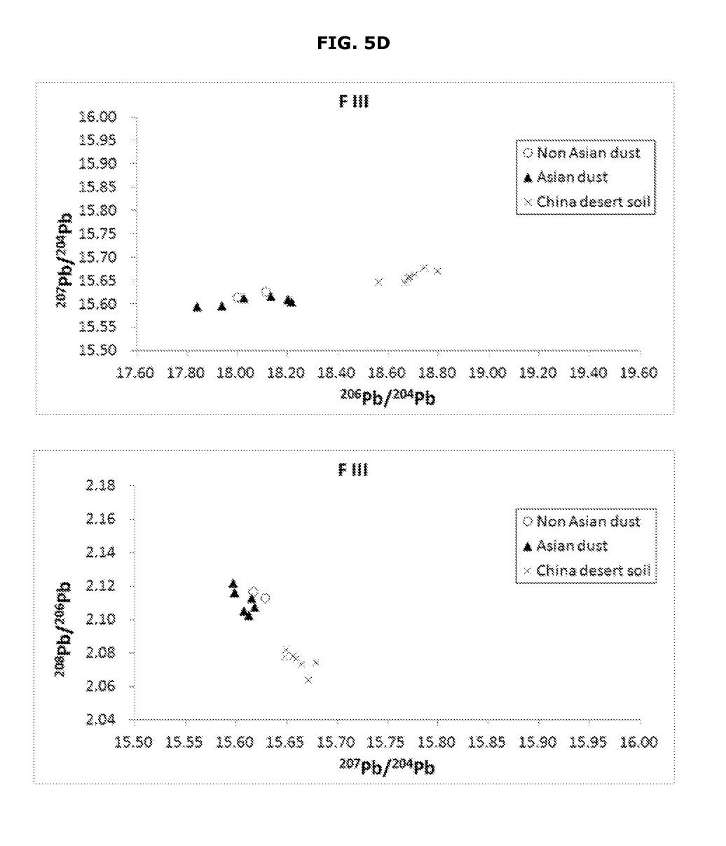

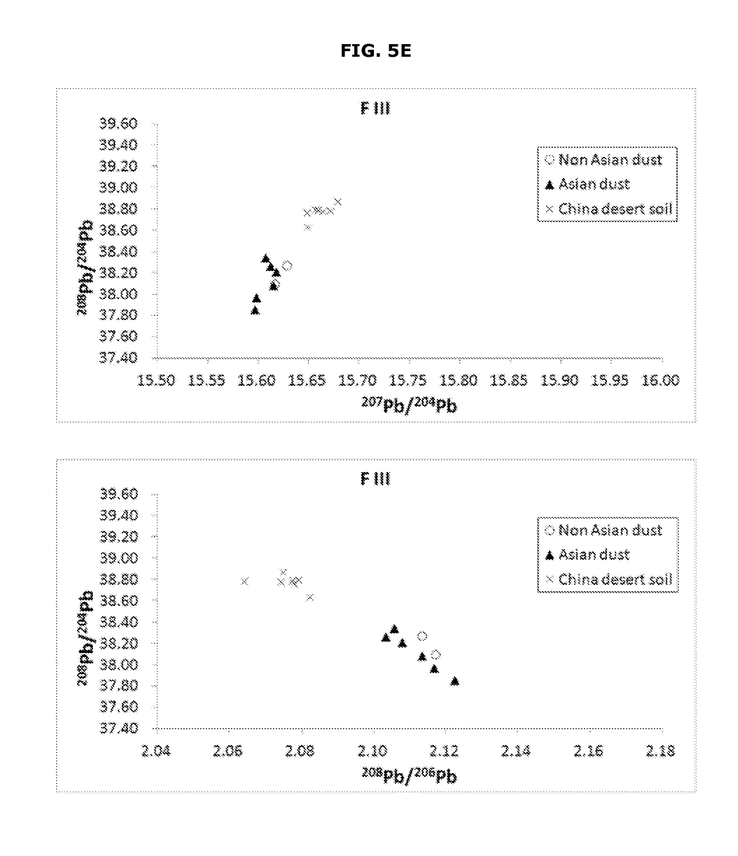

FIGS. 5A to 5E are graphs showing analysis results according to the third step in the method of resolving the heavy metal contamination source based on the sequential extraction scheme and the isotope analysis scheme according to the exemplary embodiment of the present invention.

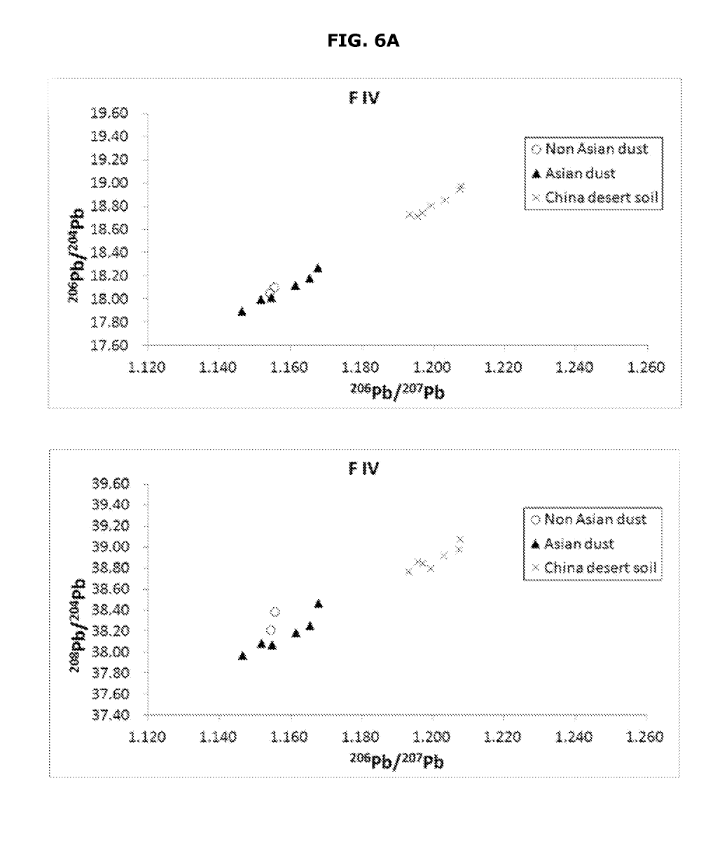

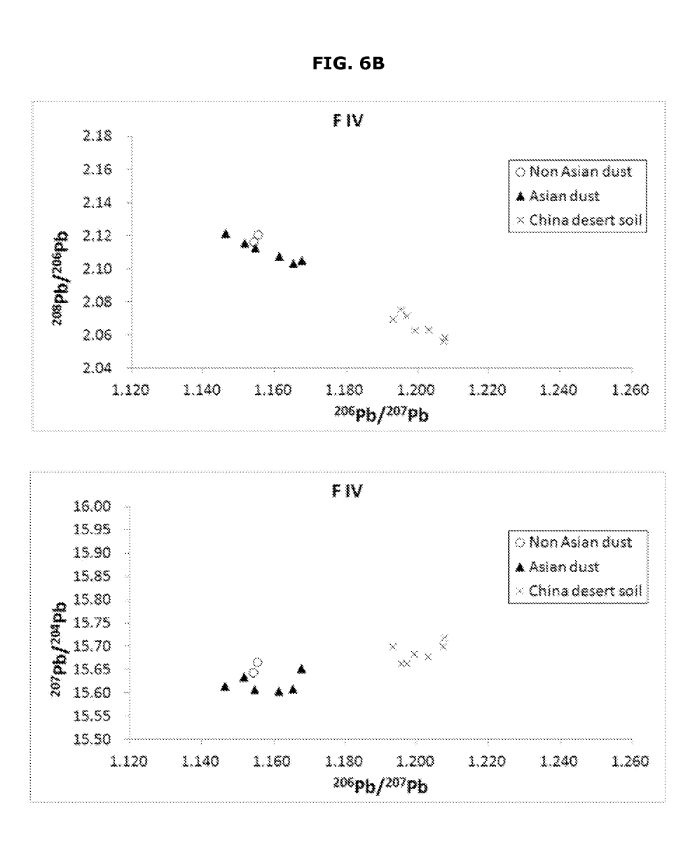

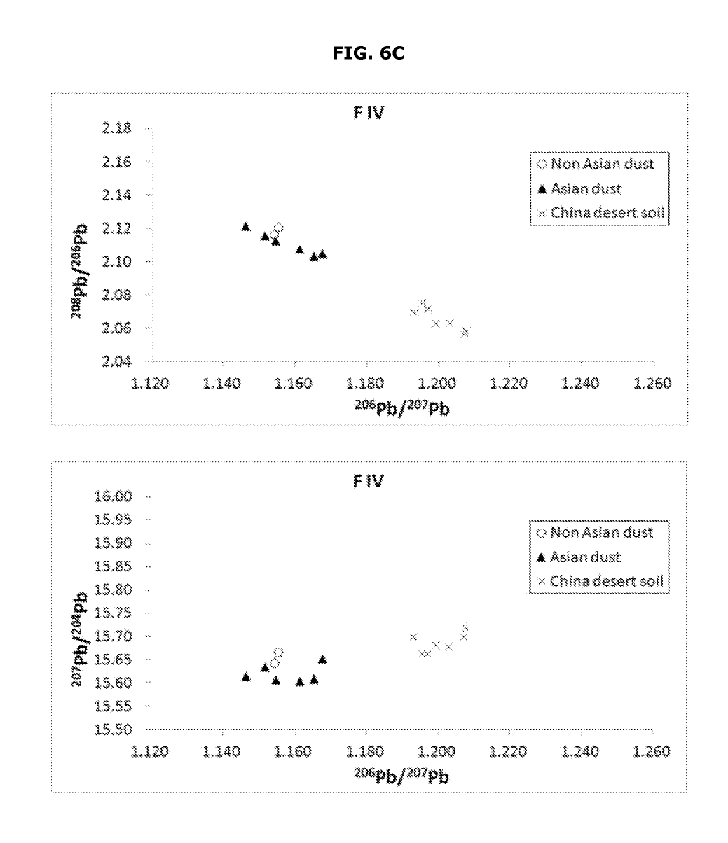

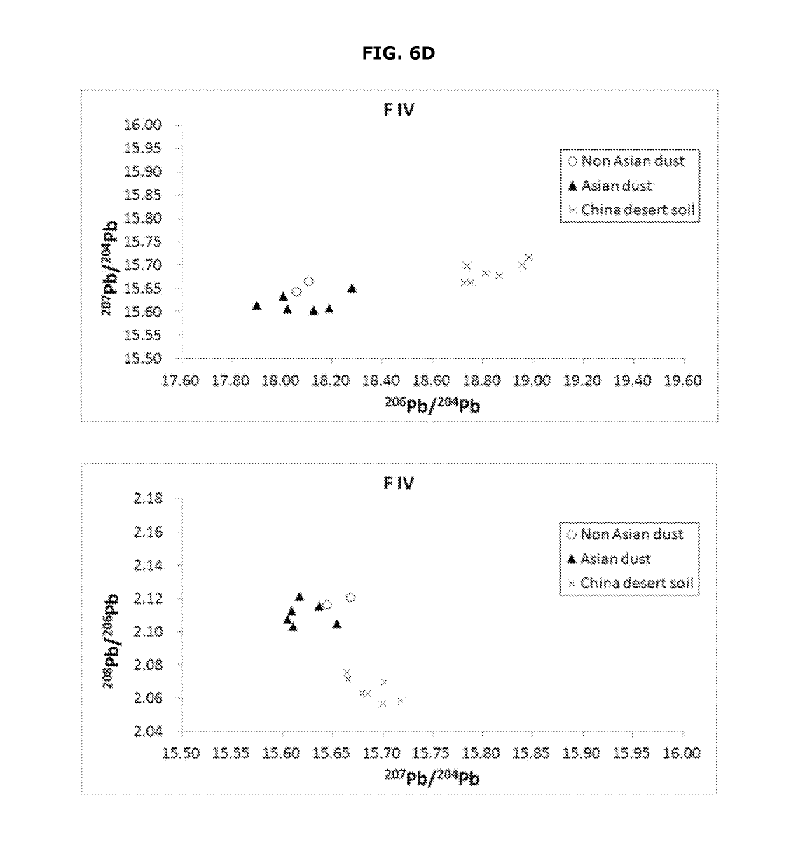

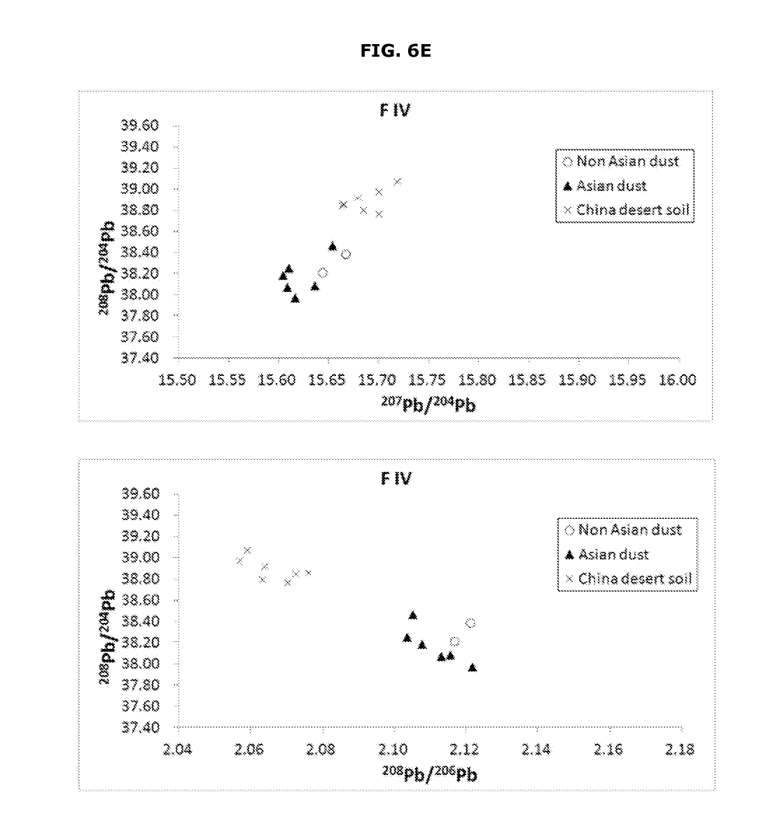

FIGS. 6A to 6E are graphs showing analysis results according to the fourth step in the method of resolving the heavy metal contamination source based on the sequential extraction scheme and the isotope analysis scheme according to the exemplary embodiment of the present invention.

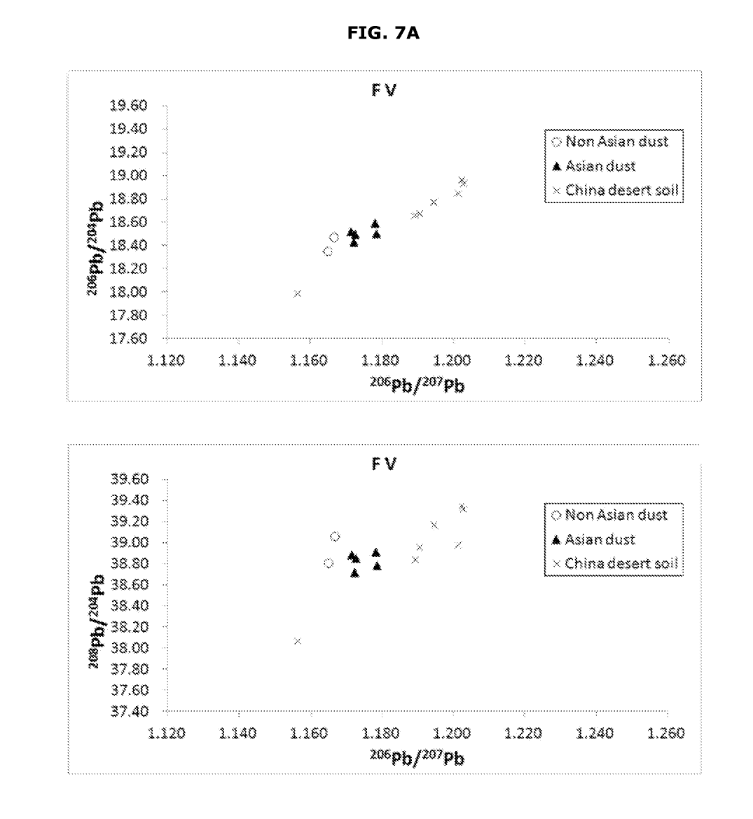

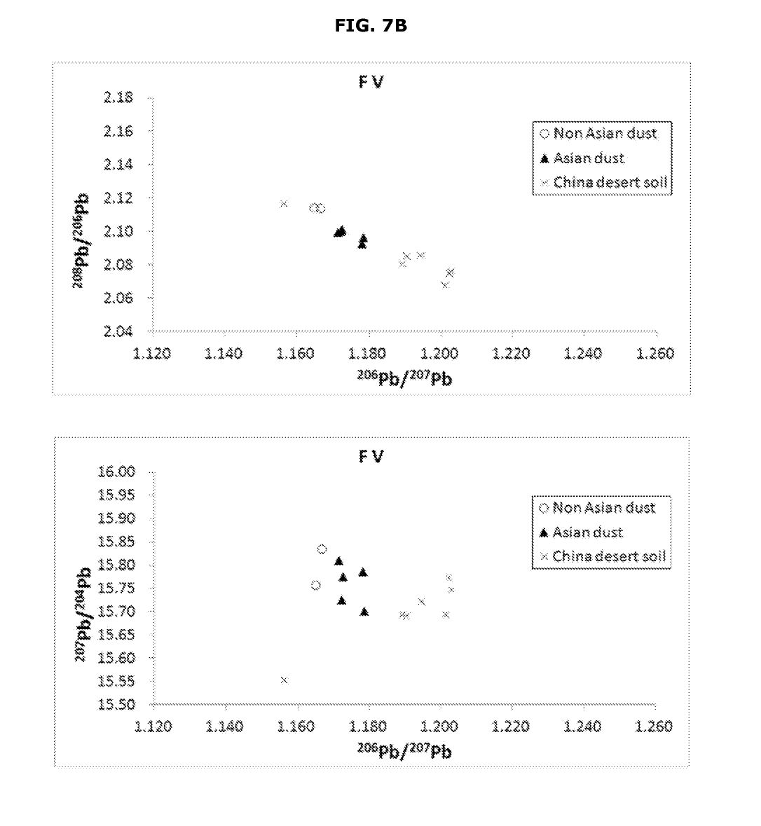

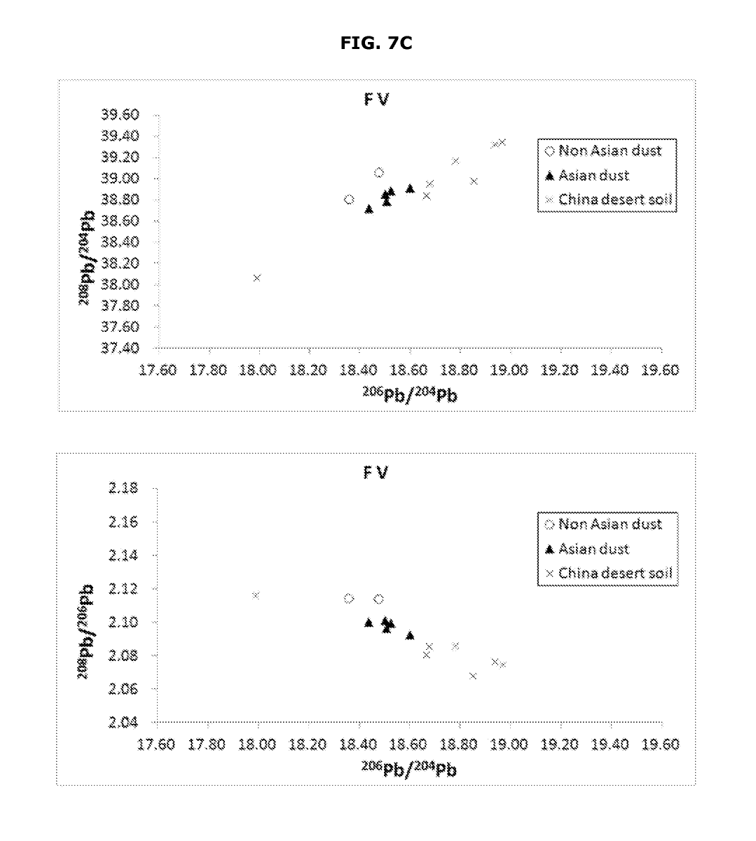

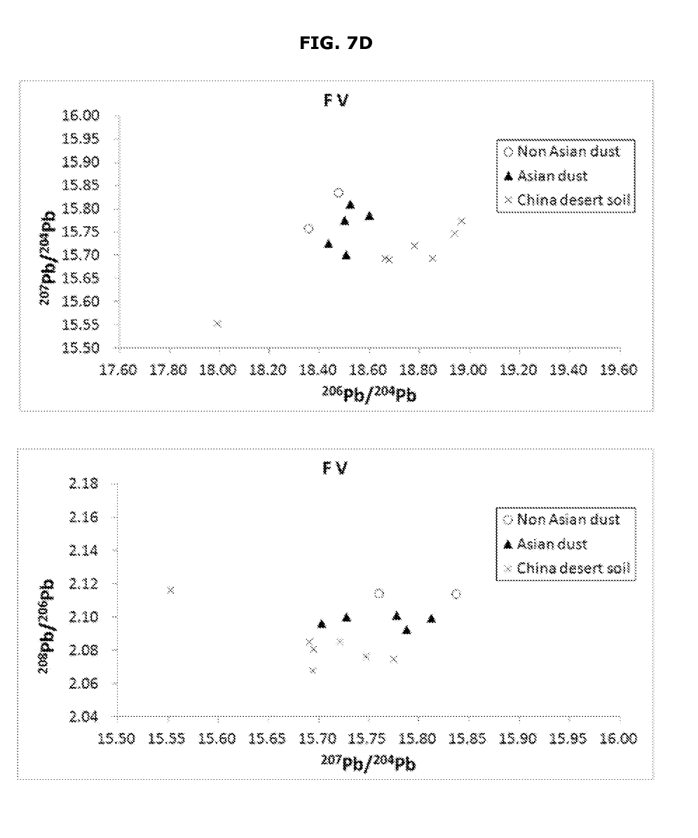

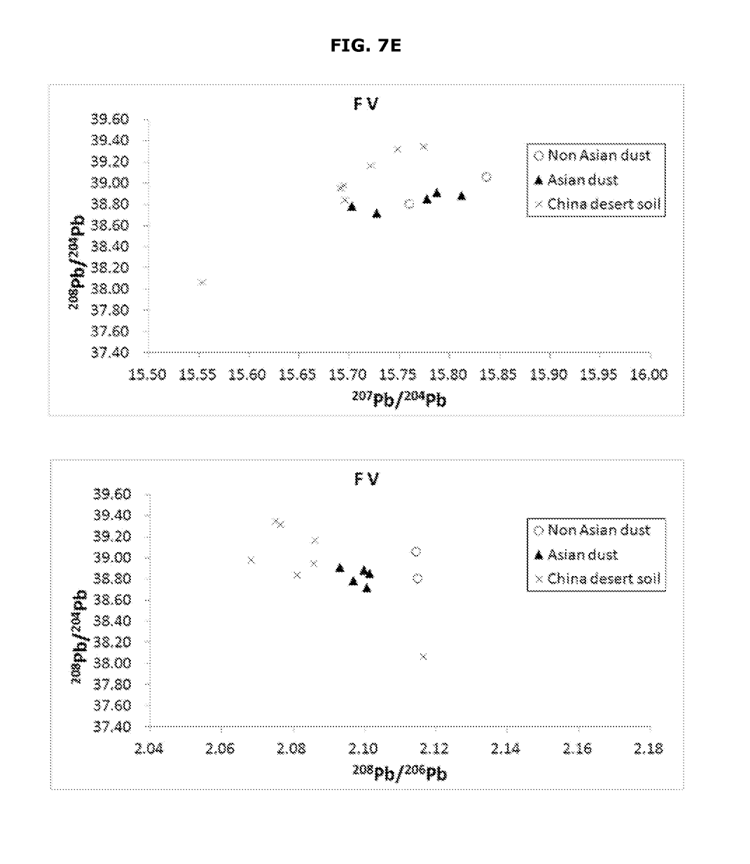

FIGS. 7A to 7E are graphs showing analysis results according to the fifth step in the method of resolving the heavy metal contamination source based on the sequential extraction scheme and the isotope analysis scheme according to the exemplary embodiment of the present invention.

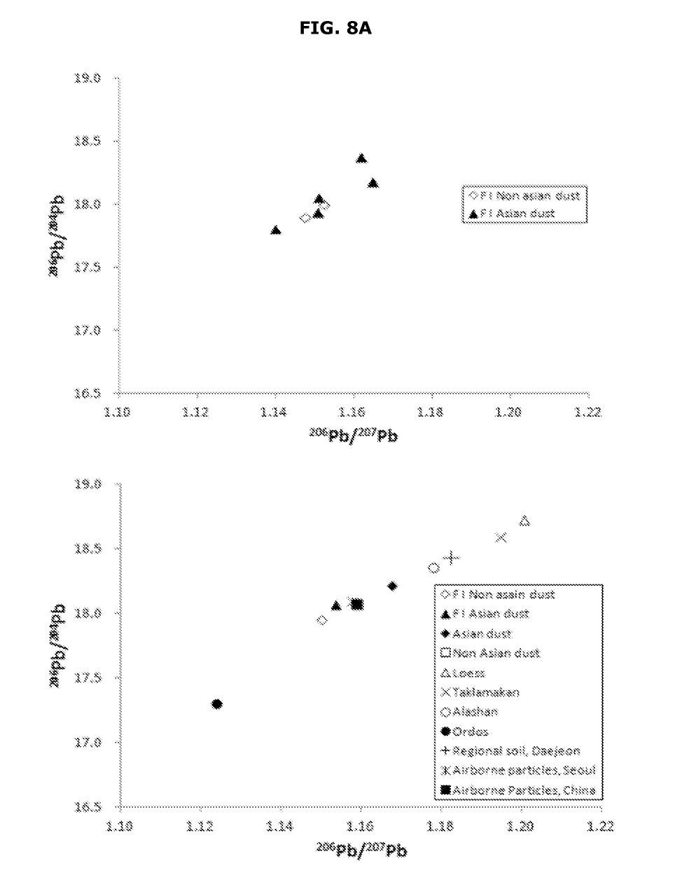

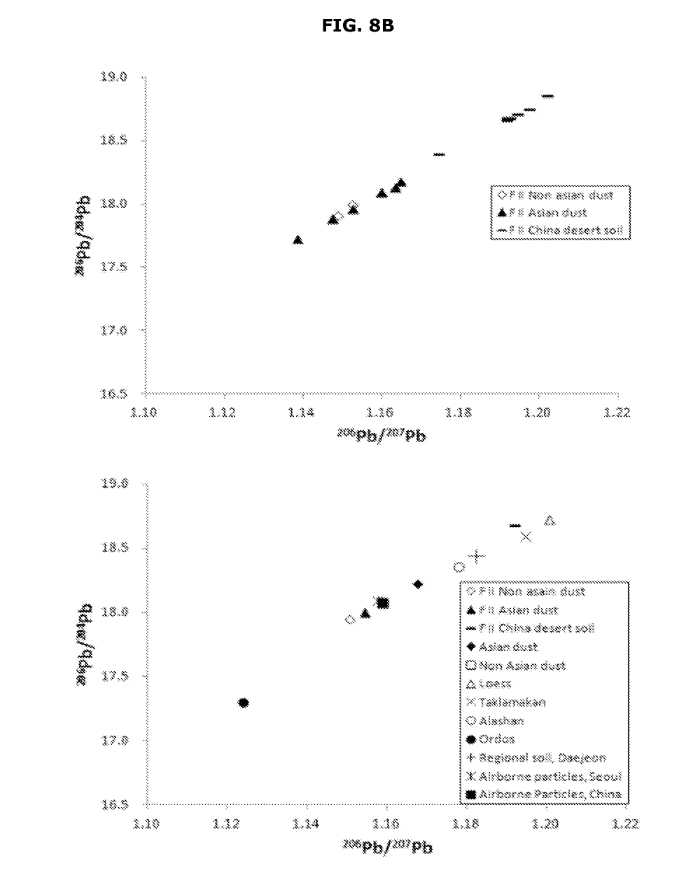

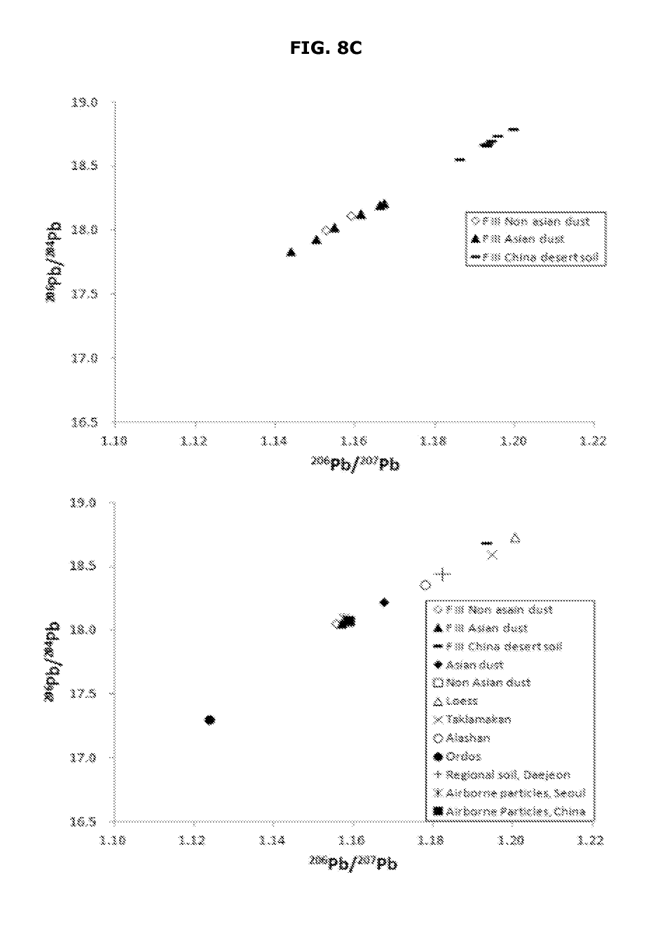

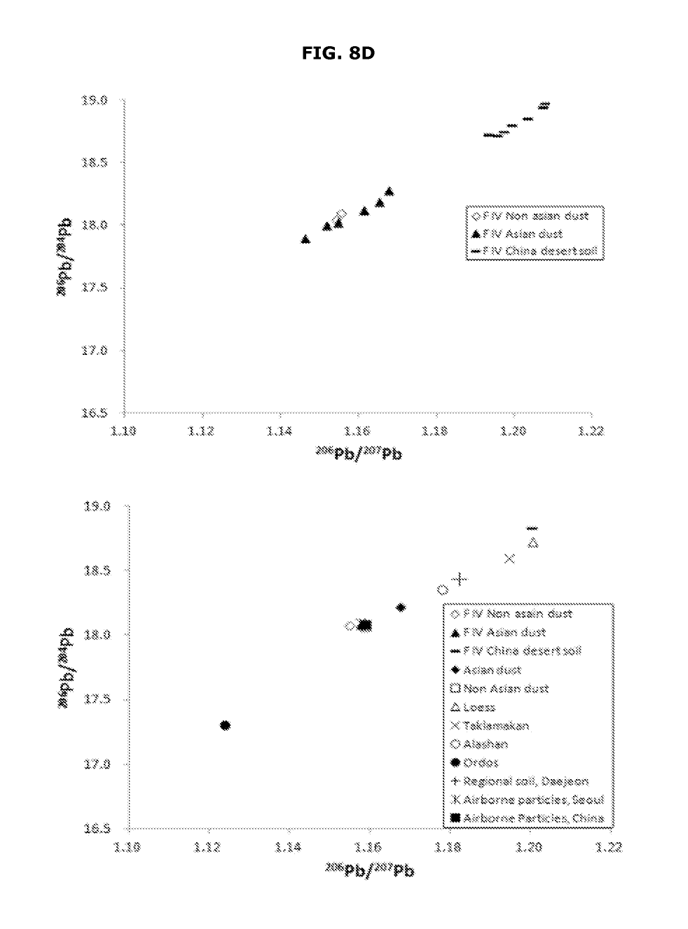

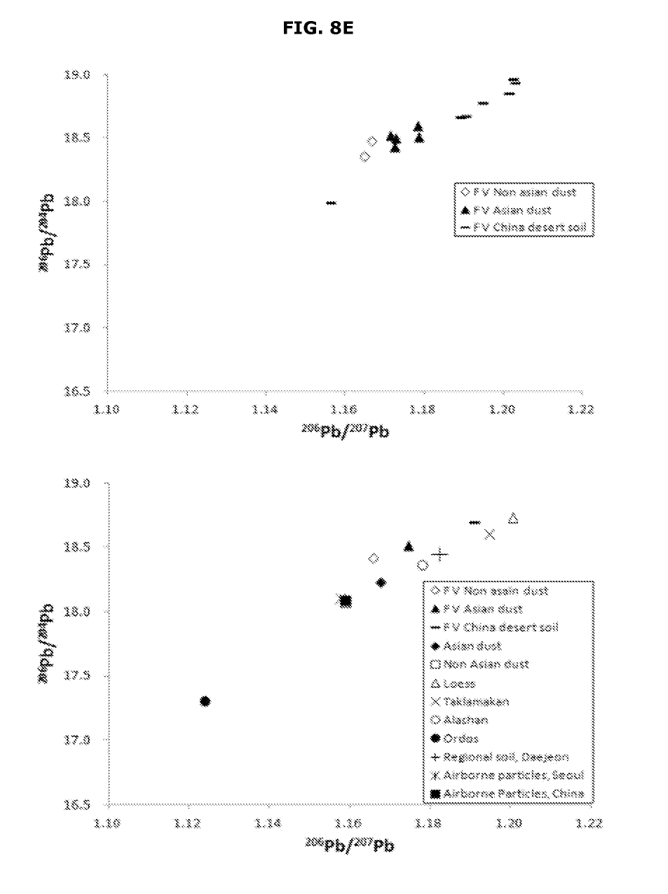

After performing the content analysis of a Pb isotope with respect to a sample subject to physical and chemical treatment through five steps (step F1 to step FV) in the sequential extraction scheme, the 10 correlations of ²⁰⁶Pb/²⁰⁴Pb vs. ²⁰⁶Pb/²⁰⁷Pb, ²⁰⁸Pb/²⁰⁴Pb vs. ²⁰⁶Pb/²⁰⁷Pb, ²⁰⁸Pb/²⁰⁴Pb vs. ²⁰⁶Pb/²⁰⁴Pb, ²⁰⁸Pb/²⁰⁶Pb vs. ²⁰⁶Pb/²⁰⁷Pb, ²⁰⁸Pb/²⁰⁶Pb vs. ²⁰⁶Pb/²⁰⁴Pb, ²⁰⁸Pb/²⁰⁴Pb vs. ²⁰⁷Pb/²⁰⁴Pb, ²⁰⁸Pb/²⁰⁴Pb vs. ²⁰⁸Pb/²⁰⁶Pb, ²⁰⁷Pb/²⁰⁴Pb vs. ²⁰⁶Pb/²⁰⁷Pb, ²⁰⁷Pb/²⁰⁴Pb vs. ²⁰⁶Pb/²⁰⁴Pb and ²⁰⁸Pb/²⁰⁶Pb vs. ²⁰⁷Pb/²⁰⁴Pb are shown in each of step F1 to step FV (see FIGS. 3A to 3E, and FIGS. 8A to 8E).

First, in the case of “cation exchange-type” (step F1 which is the first step), since the content of Pb represents a significantly low value in the China desert soil, the isotope analysis of the Pb isotope is not performed with respect to the China desert soil. Accordingly, the 10 correlation are reviewed with respect to the content of the Pb isotope in the Asian dust and the non-Asian dust except for the China desert soil (see FIGS. 3A to 3E).

Accordingly, the origin of the Asian dust (marked as ‘o’) is matched with the origin of the non-Asian dust (marked as ‘▴’) in step F1 (see the upper side of FIG. 3A).

Next, 10 correlation are reviewed in the case of “carbonate fraction” (step FII which is the second step). As a result, the contamination sources of the Asian dust and the non-Asian dust represent the same origins in all correlation, which remarkably makes a difference from the origin of Pb contained in the desert soil (China desert soil) (see FIGS. 4A to 4E).

Therefore, the contamination of heavy metal including Pb existing as “carbonate fraction” in the Asian dust and the non-Asian dust is caused by an artificial contamination source instead of the origin of the China desert soil.

In this case, among several correlations, the correlation of ²⁰⁶Pb/²⁰⁴Pb vs. ²⁰⁶Pb ²⁰⁶/²⁰⁷Pb represents the most clear linear relationship. The correlation of ²⁰⁶Pb/²⁰⁴Pb vs. ²⁰⁶Pb/²⁰⁷Pb is the most suitable correlation used to investigate the contamination source.

Next, the “Fe—Mn hydroxide-fraction” (step FIII which is the third step) represents the same result as that of the “carbonate fraction” (see FIGS. 5A to 5E).

In detail, the contamination of heavy metals such as Pb existing at the type of “Fe—Mn hydroxide-fraction” in the Asian dust and the non-Asian dust is not caused by the China desert soil. On the contrary, the heavy metal contamination of the Asian dust and the non-Asian dust is caused from the same origin.

The same result is represented in the content analysis of the Pb isotope in the “organic matters and sulfide fraction” (step FIV) (see FIGS. 6A to 6E).

It is noted from the analysis results in steps FIII and FIV when comparing with step FII that the difference in the content ratio between the Pb isotope in the China desert soil and the Pb isotope in the Asian dust is more remarkable. This represents that the contamination source can be more clearly investigated in step FIII or step FIV instead of step FII when the heavy metal contamination source is investigated by measuring the content ratio of the Pb isotope after the pre-treatment is performed through the sequential extraction scheme (see FIGS. 5A to 5E, and FIGS. 6A to 6E).

In addition, as the content ratios of the Pb isotopes contained in the Asian dust, the non Asian dust, and the China desert soil are measured and reviewed, the comparison between the content ratios of the Pb isotopes by analyzing the contents of the Pb isotopes in steps FIII to FIV through the sequential extraction scheme is the most desirable scheme to definitely specify the contamination source of the artificial Pb isotopes resulting from the industrial activity of a human being.

It should be noted that the measurement and the analysis of the content ratio of the Pb isotope in step FII can more definitely specify the contamination source of the Pb isotope when comparing with the measurement and the analysis of the content ratio of the isotope through the complete analysis of the related art.

Finally, as the content ratio of the Pb isotope is analyzed by using the “residual fraction” (step FV), the content ratios of the Pb isotopes in the Asian dust, the non-Asian dust, and the desert oil have approximate values (see FIGS. 7A to 7E).

Further, even in the correlation analysis, 10 correlations are distributed in a similar area, so that the origins of the Pb isotopes contained in the effluent in step FV are significantly similarly represented in the Asian dust, the non-Asian dust, and the China desert soil.

Therefore, according to the analysis result of the Pb isotopes contained in the Asian dust, the non Asian dust, and the China desert soil in step 5, the origins of materials constituting the Asian dust, the non Asian dust, and the desert are significantly similar to each other.

Since the content ratio of the Pb isotope in step FV is completely different from the content ratios of the Pb isotopes in steps FII, FIII, and FIV, the Pb isotope contained in the contaminated heavy metal analyzed in the Asian dust and the no-Asian dust is not come from the desert oil, but come from atmospheric pollution, in detail, the non-Asian dust.

Therefore, in order to investigate whether the Asian dust, the non Asian dust, and the China desert soil have the same origins, after the pre-treatment is performed in steps FII, FIII, and FIV through the sequential extraction scheme, a remaining sample is completely decomposed, that is, dissolved in step FV and the content ratio of the Pb isotope is measured by using the effluent. According to the above scheme, whether the Asian dust, the non-Asian dust, and the desert soil have the same origins can be easily determined.

If the Pb isotope is analyzed by completely decomposing the sample according to the related instead of using the sequential extraction scheme according to the exemplary embodiment of the present invention, since the original origin of the Pb isotope is mixed with the Pb isotope contamination source due to the atmospheric pollution resulting from the industrial activity of a human being so that the content ration of the Pb isotope may be changed. Accordingly, the

Finally, the average value in the content ratios of the Pb isotopes in the Asian dust, the non Asian dust, and the desert oil measured in each step of the sequential extraction scheme is sown in FIGS. 8A to 8E.

FIGS. 8A to 8E are graphs showing an average value of the content ratios of Pb isotopes contained in the Asian dust, the non Asian, and the China desert soil in each step of the method of resolving the heavy metal contamination source based on the sequential extraction scheme and the isotope analysis scheme according to the exemplary embodiment of the present invention.

In the graph shown in FIGS. 8A to 8E, the non-Asian dust from step F1 to step FV represents atmospheric floaters and the Asian dust represents yellow dust. In addition, the right graph shows the average value in the content ratio of a Pb isotope in each sample

The term “Loss” represents the average value of the sample collected in Chinese loess plateau, the term “Takamakan” represents the average value of the sample collected in a Taklimakan desert, the term “Alashan” represents the average value of the sample collected in an Alashan desert located in Inner Mongolia, and the term “Ordos” represents the average value of the sample collected in an Ordos desert located in the Inner Mongolia. The regions are representative sources of Asian dust to exert an influence on Korea.

Subsequently, the term “Regional soil, Daejeon” represents the average value of a soil sample collected in Daejeon of Korea, the term “Airborne particles, Seoul” represents the average value of the non-Asian dust in Seoul of Korea, and the term “Airborne particles, China” represents the average value of the non-Asian dust samples collected in 20 cities of representative industrial cities and big cities. In this case, the term “Airborne particles, China” is an average value obtained by analyzing materials laid down in various cited references.

Meanwhile, as shown in FIGS. 8A to 8E, the average value in the content ratios of the Pb isotopes contained in the Asian dust and the non-Asian dust measured in step F1 represent a value approximate to the average value in the content ratio of the Pb isotope measured in the big city and the industrial region of China.

In addition, although the average value in the content ratios of the Pb isotopes contained in the Asian dust and the non-Asian dust measured in step FII is not equal to the average value in the content ratios of the Pb isotopes measured in the big cities and the industrial region of China, the average value in the content ratios of the Pb isotopes contained in the Asian dust and the non-Asian dust measured in step FII represents a value approximate to the average value in the content ratios of the Pb isotopes measured in the big cities and the industrial regions of China.

In addition, the average value in the content ratio of the Pb isotope contained in the China desert soil measured in step FII represent a value approximate to the average value in the content ratio of the Pb isotope contained in the Taklimakan desert soil.

In addition, the average value in the content ratio of the Pb isotope contained in the Asian dust and the non-Asian dust measured in step FIII represent a value substantially equal to the average value in the content ratio of the Pb isotope measured in the big cities and the industrial cities of China.

According to the above tendency, the average value in the content ratio of the Pb isotope of the Asian dust and the non-Asian dust measured in step FIV is completely equal to the average value in the content ratio of the Pb isotope measured in the big cities and the industrial cities of China.

Further, it can be understood from the result that the heavy metal elements such as Pb dissolved in steps FIII and FIV, that is, a contamination source contaminated at the Fe—Mn hydroxide, an organic matter, and a sulfide fraction are come from the atmospheric pollution.

In addition, it can be understood from the result that the contamination source of heavy metal isotopes such as Pb existing at the ion-exchange type and the carbonate fraction type is come from the atmospheric pollution occurring in the big cities and the industrial cities of China.

Meanwhile, the origin of the Pb isotope contained in the China desert soil dissolved in steps FIII and FIV may be Taklimakan and Loess with high possibility.

Finally, regarding the average value in the content ratio of the Pb isotope dissolved in step FV and eluted, the average value in the content ratio of the Pb isotope contained in the Asian dust and the China desert soil represents a value approximate to the average value in the content ratio of the Pb isotope contained in the Alashan desert soil, and the average value in the content ratio of the Pb isotope contained in the China desert soil represents a value approximate to the average value in the content ratio of the Pb isotope contained in Taklimakan.

Although a method of resolving a heavy metal contamination source based on a sequential extraction scheme and an isotope analysis scheme according to exemplary embodiments of the present invention have been described for the illustrative purpose, it is understood that the present invention should not be limited to these exemplary embodiments but various changes, modifications, equivalents can be made by one ordinary skilled in the art within the spirit and scope of the present invention as hereinafter claimed.

What is claimed is:

1. A method of resolving a heavy metal contamination source based on a sequential extraction scheme and an isotope analysis scheme, the method comprising:
(A) preparing a first sample including a Pb isotope;
(B) preparing a first solution comprising 1M MgCl₂ (pH=7), introducing the first sample into the first solution, and stirring the first solution at a normal temperature for one hour to retrieve a second solution;
(C) preparing a third solution comprising 1M CH₃COONa, adjusting a pH of the third solution to 5 by using HOAc, introducing a second sample, and stirring a result at the normal temperature for five hours to obtain a fourth solution and retrieve a third sample which is not dissolved;
(D) preparing a fifth solution comprising 0.04M NH₂OH.HCl+25% HOAc, adjusting a pH of the fifth solution to 2, introducing the third sample, and heating the result at the temperature of 96° C. for six hours to obtain a sixth solution and retrieve a fourth sample which is not dissolved;
(E) preparing a seventh solution comprising 30% H₂O₂+0.02M HNO₃, introducing the fourth sample, heating a result at the temperature of 85° C. for five hours, cooling the result, additionally introducing an eighth solution comprising 3.2M NH₄OAc+20% HNO₃, and stirring a result at the normal temperature for 30 minutes to obtain a ninth solution;
(F) introducing the fifth sample into a tenth solution comprising HF+HClO₄, evaporating a result at a temperature of 110° C. to completely dry the result, introducing the result into 12M HCl solution, heating the result for 30 minutes, and completely dissolving the fifth sample to obtain an eleventh solution; and
(G) analyzing a content of the Pb isotope contained in a Pb isotope effluent of the second solution, the fourth solution, the sixth solution, the ninth solution, and the eleventh solution obtained in steps (B) to (F).

2. The method of claim 1, wherein the Pb isotope comprises ²⁰⁴Pb, ²⁰⁶Pb, ²⁰⁷Pb, and ²⁰⁸Pb.

3. The method of claim 2, wherein the Pb isotope used in the analyzing of the content of the Pb isotope in step (G) comprises ²⁰⁴Pb, ²⁰⁶Pb, and ²⁰⁷Pb.

4. The method of claim 3, wherein a contamination source of the Pb isotope comprising ²⁰⁴Pb, ²⁰⁶Pb, and ²⁰⁷Pb is specified from a correlation between a ratio of ²⁰⁶Pb/²⁰⁴Pb obtained from a content of ²⁰⁶Pb and a content of ²⁰⁴Pb, and ratio of ²⁰⁶Pb/²⁰⁷Pb obtained from a content of ²⁰⁶Pb and a content of ²⁰⁷Pb in the Pb isotope.

5. The method of claim 1, wherein the sample is pulverized in a particle size of 80 meshes to 100 meshes.

6. A method of resolving a heavy metal contamination source based on a sequential extraction scheme and an isotope analysis scheme, the method comprising:
(A) preparing a first sample including a Pb isotope;
(B) preparing a first solution comprising 1M MgCl₂ (pH=7), introducing the first sample into the first solution, and stirring the first solution at a normal temperature for one hour to retrieve a second solution;
(C) preparing a third solution comprising 1M CH₃COONa, adjusting a pH of the third solution to 5 by using HOAc, introducing a second sample, and stirring a result at the normal temperature for five hours to obtain a fourth solution and retrieve a third sample which is not dissolved;
(D) preparing a fifth solution comprising 0.04M NH₂OH.HCl+25% HOAc, adjusting a pH of the fifth solution to 2, introducing the third sample, and heating a result at the temperature of 96° C. for six hours to obtain a sixth solution and retrieve a fourth sample which is not dissolved;
(E) preparing a seventh solution comprising 30% H₂O₂+0.02M HNO₃, introducing the fourth sample, heating a result at the temperature of 85° C. for five hours, cooling the result, additionally introducing an eighth solution comprising 3.2M NH₄OAc+20% HNO₃, and stirring a result at the normal temperature for 30 minutes to obtain a ninth solution; and
(F) analyzing a content of the Pb isotope contained in a Pb isotope effluent of the sixth solution obtained in step (D) or the ninth solution obtained in step (E),
wherein a Pb isotope contamination source resulting from an industrial activity is specified in step (F).

7. The method of claim 6, wherein the Pb isotope comprises ²⁰⁴Pb, ²⁰⁶Pb, ²⁰⁷Pb, and ²⁰⁸Pb.

8. The method of claim 7, wherein the Pb isotope used in the analyzing of the content of the Pb isotope in step (F) comprises ²⁰⁴Pb, ²⁰⁶Pb, and ²⁰⁷Pb.

9. The method of claim 8, wherein a contamination source of the Pb isotope comprising ²⁰⁴Pb, ²⁰⁶Pb, and ²⁰⁷Pb is specified from a correlation between a ratio of ²⁰⁶Pb/²⁰⁴Pb obtained from a content of ²⁰⁶Pb and a content of ²⁰⁴Pb, and a ratio of ²⁰⁶Pb/²⁰⁷Pb obtained from a content of ²⁰⁶Pb and a content of ²⁰⁷Pb in the Pb isotope.

10. The method of claim 6, wherein the sample is pulverized in a particle size of 80 meshes to 100 meshes.
